SEQUENCE LISTING (2 sequences)

SEQ ID NO: 1 - DNA, 1129 bp, Botryococcus sudeticus AVFF007
  misc_feature: FERM BP-11420
tacggtcgcttcggtgagtactgctatggccttcctttctacgggggacgggctcctgggattaacttctcgggatccggattccgtgatgatactttgagtaaattggagtgttcaaagcaagcctgcgctctgaacattttagcatggaatatcacgataggactctggcctatcttgttggtctgtaggaccagagtaatgattaagagggacagtcgggggcattcgtatttcattgtcagaggtgaaattcttggatttatgaaagacgaactactgcgaaagcatttgccaaggatgttttcattaatcaagaacgaaagttgggggctcgaagacgattagataccgtcgtagtctcaaccataaacgatgccgactagggattagcagacgtttcattgatgactctgctagcaccttatgtgaaaacaaagtttttgggttccggggggagtatggtcgcaaggctgaaacttaaaggaattgacggaagggcaccaccaggcgtggagcctgcggcttaatttgactcaacacgggaaaacttaccaggtccagacatagtgaggattgacagattgagagctctttcttgattctatgggtggtggtgcatggccgttcttagttggtgggttgccttgtcaggttgattccggtaacgaacgagacctcagctcctaaatagtccatgccgccttttggcggtatgctgacttcttagagggactattggcgttctagccaatggaagtatgaggcaataacaggtctgtgatgcccttagatgttctgggccgcacgcgcgctacactgatgcgttcaacgagcctatccttgaccgagaggtccgggtaatctttgaaaccgcatcgtgatggggctagactattgcaattattagtctttaacgaggaatgcctagtaagcgcgattcatcagatcgcgttgattacgtccctggtacacaccgcccgtcgctcctaccgattgggtgtgctggtgaagtgttcggatcggcaaagctgggggcaactcttgcattgccgagaagttcattaaaccctcccacctagaggaaggagaagtcgtaacaaggtttccgtaggtgaacctgcggaag

SEQ ID NO: 2 - DNA, 1650 bp, Chlorococcum sp. FFG039
  misc_feature: FERM BP-22262
ctgcttatacggtgaaactgcgaatggctcattaaatcagttatagtttatttgatggtacctcctacttggataaccgtaggaaatctagagctaatacatgcgtaaatcccgacttctggaagggacgtatttattagataaaaggccagccgggcttgcccgaccttaggcgaatcatgataacttcacgaatcgcatggccttgtgccggcgatgtttcattcaaatttctgccctatcaactttcgatggtaggatagaggcctaccatggtggtaacgggtgacggaggattagggttcgattccggagagggagcctgagaaacggctaccacatccaaggaaggcagcaggcgcgcaaattacccaatcccgacacggggaggtagtgacaataaataacaatactgggcatttatgtctggtaattggaatgagtacaatgtaaatatcttaacgagtatccattggagggcaagtctggtgccagcagccgcggtaattccagctccaatagcgtatatttaagttgttgcagttaaaaagctcgtagttggatttcgggtgcgtcgacgcggtctgcctctggtatgtactgcgctcggtgcacctttcagctgaatacgctctcctgggcttaactgcttgggaagtggagtcagcagagtgaccttgagcaaacaagagtgttcaaagcaagccgacgctctgaattttttagcatggaatcacacgataggactctggcctatcttgttggtctgtaggaccggagtaatgattaagagggacagtcgggggcattcgtatttcattgtcagaggtgaaattcttggatttatgaaagacgaacttctgcgaaagcatttgccaaggatgttttcattgatcaagaacgaaagttgggggctcgaagacgattagataccgtcgtagtctcaaccataaacgatgccgactagggattggcaggtgttccattgatgaccctgccagcaccttatgagaaatcaaagtttttgggttccggggggagtatggtcgcaaggctgaaacttaaaggaattgacggaagggcaccaccaggcgtggagcctgcggcttaatttgactcaacacggggaaacttaccaggtccagacacggggaggattgacagattgagagctctttcttgattctgtgggtggtggtgcatggccgttcttagttggtgggttgccttgtcaggttgattccggtaacgaacgagacctcagcctgctaaatagtcacgggtaccttggtacacgcctgacttcttagagggactattggcgtctagtcaatggaagtgtgaggcaataacaggtctgtgatgcccttagatgttctgggccgcacgcgcgctacactgatgcattcaacgagcctatccttggccgagaggtccgggtaatctttgaaactgcatcgtgatggggatagattattgcaattattagtcttcaacgaggaatgcctagtaagcgcaagtcatcagcttgcgttgattacgtccctgccctttgtacacaccgcccgtcgctcctaccgattgggtgtgctggtgaagtgttcggattggctccagggatgggcaaccactcctgttgctgagaagaac

(19) United States
(12) Patent Application Publication
(10) Pub. No.: US 2016/0194671 A1
(43) Pub. Date: Jul. 7, 2016

(54) CULTURE METHOD FOR MICROALGAE THAT IMPROVES OIL CONTENT RATIO, METHOD FOR MANUFACTURING ALGAL BIOMASS, AND NOVEL MICROALGA

(71) Applicant: FUJIFILM Corporation, Tokyo (JP)

(72) Inventors: Hideyuki KANEHARA, Ashigarakami-gun (JP); Tadashi MATSUNAGA, Tokyo (JP); Tsuyoshi TANAKA, Tokyo (JP); Masayoshi TANAKA, Tokyo (JP); Masaki MUTO, Tokyo (JP)

(73) Assignee: FUJIFILM Corporation, Tokyo (JP)

(21) Appl. No.: 15/074,928

(22) Filed: Mar. 18, 2016

Related U.S. Application Data
(63) Continuation of application No. PCT/JP2014/074959, filed on Sep. 19, 2014.

(30) Foreign Application Priority Data
Sep. 20, 2013 (JP) 2013-194973
Sep. 20, 2013 (JP) 2013-194977
Mar. 20, 2014 (JP) 2014-058126

(51) Int. Cl. C12P 7/64; C12R 1/89; C12N 1/12

(52) U.S. Cl. CPC C12P 7/64; C12N 1/12; C12R 1/89

(57) ABSTRACT

In a liquid surface-floating culture method in which culturing of microalgae is performed on the liquid surface, there is provided a culture method in which the oil content in microalgae is improved. In addition, there is a provided a method for decreasing the probability of recovery of bottom surface algae. Furthermore, an object of the present invention is to provide a culture method in which the proliferation rate of microalgae is improved.

Culturing is performed such that a medium is suctioned from a region, in which there is a small quantity of microalgae between the liquid surface and the bottom surface, is discarded, and is replaced with a medium of which the concentration of a nitrogen compound or a phosphorus compound is lower than that of the above-described medium. In addition, a liquid is added thereto immediately before collecting microalgae on the liquid surface, and the water depth in a culture vessel is made to be deep. In addition, the microalgae are subjected to liquid surface-floating culture using a medium containing sugar. In the present invention, microalgae which can form a biofilm on the liquid surface and have at least one characteristic selected from the group consisting of the following (1) to (8) when being cultured in a medium within a culture vessel may be used. (1) The sum of the quantity of algal bodies of microalgae existing on the liquid surface and in a region from 1 cm below the liquid surface to the liquid surface, and the quantity of algal bodies of microalgae on the bottom surface of a culture vessel is greater than or equal to 10 times the quantity of algal bodies existing in the other region within the culture vessel; (2) the specific gravity of microalgae on the liquid surface is smaller than that of microalgae on the bottom surface of the culture vessel; (3) the specific gravity of microalgae on the liquid surface is greater than that of water; (4) the oil content of microalgae on the liquid surface is higher than that of microalgae on the bottom surface; (5) the size of microalgae on the liquid surface is larger than that of microalgae on the bottom surface; (6) a formed biofilm includes a film-like outer layer and an inner layer which has a plurality of bubble-like structures, and the outer layer is thicker than the inner layer; (7) a part of a formed biofilm has a pleat-like structure in a medium; and (8) in a case where microalgae obtained by collecting a biofilm which has been formed and subjecting the collected biofilm to suspension treatment are seeded on the liquid surface of a medium, the microalgae can be deposited in the medium.

### CROSS-REFERENCE TO RELATED APPLICATIONS

This application is a Continuation of PCT International Application No. PCT/JP2014/074959 filed on Sep. 19, 2014, which claims priority under 35 U.S.C §119(a) to Japanese Patent Application No. 2013-194973 filed on Sep. 20, 2013, Japanese Patent Application No. 2013-194977 filed on Sep. 20, 2013 and Japanese Patent Application No. 2014-058126 filed on Mar. 20, 2014. Each of the above application(s) is hereby expressly incorporated by reference, in its entirety, into the present application.

### BACKGROUND OF THE INVENTION

1. Field of the Invention

The present invention relates to a liquid surface-floating culture method for microalgae that can improve the biomass content ratio in a collected substance by improving the composition of a medium.

2. Description of the Related Art

In general, culturing of microalgae is performed while the microalgae are dispersed in a medium (hereinafter, referred to as dispersion culture). However, in such a culture method, an energy source is required in order to perform stirring, and a centrifugal separator, a flocculant, or the like is required in order to collect microalgae which have been dispersed. For this reason, although microalgae containing oil, which have a possibility of being applicable to fuel on the inside and the outside of a bacterial cell, are found, there is still no successful example of commercialization thereof, since costs of culturing and collecting microalgae increase significantly.

An effect of the nitrogen content in a medium on growth of algae or production of a substance has been reported in the World Journal of Microbiology and Biotechnology 8, 121-124, 1992 and Journal of Applied Phycology (2005) 17: 309-315. In addition, as a specific example of application of algae on production of a substance, a method of producing euglena containing wax ester, which includes a first step of aerobically culturing microalga euglena, a second step of further culturing a medium in which the above-described microalga euglena is cultured, as a nitrogen-starved condition, and a third step of retaining a cell of microalga euglena under an anaerobic condition, is disclosed in JP2012-023977A. In addition, green alga Scenedesmus, in which fatty acid-based hydrocarbons accumulate within an algal body in a culture solution of which the nitrogen content is greater than or equal to a certain value, is disclosed in JP2012-044923A.

### SUMMARY OF THE INVENTION

As a method for improving productivity of useful substances derived from microalgae, changing of the composition of a medium in the middle of culturing (hereinafter, also called medium replacement) is performed. However, dispersion culture is employed in the most of culturing of microalgae, and it is impossible to perform medium replacement if alga body concentration such as filtration or centrifugation of alga body dispersion liquid is not performed since a medium and microalgae exist in the same space. However, in such a process, there are problems in that an expensive apparatus is required, the process is extremely complicated, and energy to be input is large. For this reason, only production of expensive useful substances has been established as an industry. An object of the present invention is to provide a method of improving productivity of useful substances by performing medium replacement without introducing an expensive apparatus such as a filtration apparatus or a centrifugal separator and without using a large amount of energy to be input, through a simple process.

In the case of the liquid surface-floating culture method, it is possible to further reduce the amount of water used, by reducing the amount of medium, compared to the dispersion culture method, and from this viewpoint, it is possible to perform culturing at low costs. However, if the water depth of a medium is too shallow, in a case where collection is performed through a deposition method, in some cases, a second substrate for collecting a microalgal biofilm on the liquid surface comes into contact with the bottom surface of a culture vessel, and collects or peels off a part of bottom surface algae. It was found that there was a possibility that this might cause a reduction in the oil content in a collected substance and also might adversely affect culturing in a case where the bottom surface algae were used as seed algae. The present invention is also designed to solve such problems. In many cases, the oil content in bottom surface algae is generally lower than that in water surface algae.

Furthermore, there are problems in that, in a case of discharging the medium in the culture vessel to the outside of the culture vessel, in many cases, the microalgal biofilm on the liquid surface adheres to the wall surface of the culture vessel; the liquid surface moves accompanied by medium replacement; the microalgal biofilm adheres to an unexpected place of the surface of the culture vessel; and the biofilm is destroyed, and as a result, difficulty of collecting the biofilm increases and the quantity of algal bodies during the collection decreases.

Another object of the present invention is to achieve more effective culturing using a substance, other than carbon dioxide, as a carbon source of which the diffusion rate in a medium is low, in liquid surface-floating culture of microalgae.

The present invention provides the following.

[1] A culture method of microalgae, which is a liquid surface-floating culture method of microalgae and has properties of producing useful substances, the method including:

a step of culturing microalgae in a medium within a culture vessel and forming a biofilm on the liquid surface of the medium; and

a step of changing the concentration of at least one component contained in the medium,

in which the useful substances produced by microalgae were increased by changing the concentration of the component.

[2] The culture method according to [1],

in which the step of changing the concentration of the at least one component contained in the medium is performed by adding a liquid having a different composition from that of the medium, into the culture vessel.

[3] The culture method according to [1],

in which the step of changing the concentration of the at least one component contained in the medium is performed by removing a part or the entirety of the medium within the culture vessel and adding a liquid having a different composition from that of the medium.

[4] The culture method according to any one of [1] to [3],

in which the step of changing the concentration of the at least one component contained in the medium is performed by decreasing the concentration of a component containing nitrogen or phosphorus.

[5] The culture method according to any one of [2] to [4],

in which the removal or the addition of the medium refers to removal of the medium between a biofilm on the liquid surface and the bottom surface of the culture vessel or addition of the liquid having a different composition.

[6] The culture method according to any one of [1] to [5],

in which, in the step of changing the concentration of the at least one component contained in the medium, the biofilm formed on the liquid surface is not removed.

[7] A culture method of microalgae which is a liquid surface-floating culture method of microalgae and has properties of producing useful substances, the method including:

a step of culturing microalgae in a medium within a culture vessel and forming a biofilm on the liquid surface of the medium;

a step of adding a liquid into the culture vessel; and

a step of collecting the biofilm from the culture vessel to which the liquid is added and the water depth of which is increased.

[8] The culture method according to any one of [1] to [7], further including:

performing treatment of peeling an adhesion site between the biofilm and the inner wall of the culture vessel.

[9] A culture method of microalgae,

in which, in a liquid surface-floating culture method in which culturing of microalgae can be performed on the liquid surface, the culturing is performed using a medium containing sugar.

[10] The culture method according to any one of [1] to [9],

in which the medium contains sugar that can be assimilated by microalgae, and at this time, the sugar that can be assimilated by microalgae is any one selected from the group consisting of monosaccharide, which is pentose or hexose, disaccharide, trisaccharide, and polysaccharide.

[11] The culture method according to [9] or [10],

in which the concentration of sugar in the medium is greater than or equal to 1 mg/mL.

[12] The culture method according to any one of [9] to [11],

in which a medium containing glucose is used.

[13] The culture method according to any one of [1] to [12],

in which the microalgae are green algae.

[14] The culture method according to any one of [1] to [13],

in which the microalgae belong to Botryococcus sp., Chlamydomonas sp., Chlorococcum sp., Chlamydomonad sp., Tetracystis sp., Characium sp., or Protosiphon sp.

[15] The culture method according to any one of [1] to [14],

in which the microalgae belong to the same species as that of Botryococcus sudeticus or Chlorococcum sp. FERM BP-22262.

[16] The culture method according to any one of [1] to [15],

in which the microalgae are Botryococcus sudeticus FERM BP-11420 or microalgae strains having taxonomically the same properties as those of Botryococcus sudeticus FERM BP-11420, or are Chlorococcum sp. FERM BP-22262 or microalgae strains having taxonomically the same properties as those of Chlorococcum sp. FERM BP-22262.

[17] A method for manufacturing algal biomass, including:

a culture step including the culture method according to any one of [1] to [16]; and

a step of collecting a formed biofilm.

[18] The manufacturing method according to [17],

in which the algal biomass is oil.

[19] Microalgae which can form a biofilm on the liquid surface and have at least one characteristic selected from the group consisting of the following (1) to (8) when being cultured in a medium within a culture vessel:

(1) the sum of the quantity of algal bodies of microalgae existing on the liquid surface and in a region from 1 cm below the liquid surface to the liquid surface, and the quantity of algal bodies of microalgae on the bottom surface of a culture vessel is greater than or equal to 10 times the quantity of algal bodies existing in the other region within the culture vessel;

(2) the specific gravity of microalgae on the liquid surface is smaller than that of microalgae on the bottom surface of the culture vessel;

(3) the specific gravity of microalgae on the liquid surface is greater than that of water;

(4) the oil content of microalgae on the liquid surface is higher than that of microalgae on the bottom surface;

(5) the size of microalgae on the liquid surface is larger than that of microalgae on the bottom surface;

(6) a biofilm to be formed includes a film-like outer layer and an inner layer which has a plurality of bubble-like structures, and the outer layer is thicker than the inner layer;

(7) a part of a biofilm to be formed has a pleat-like structure in a medium; and

(8) in a case where microalgae obtained by collecting a formed biofilm and subjecting the collected biofilm to suspension treatment are seeded on the liquid surface of a medium, the microalgae can be deposited in the medium.

[20] The microalgae according to [19],

in which microalgae are the microalgae defined by any one of [13] to [16].

[21]A method of culturing microalgae capable of forming a biofilm on the liquid surface,

in which microalgae used are the microalgae according to [19] or [20].

[22] The culture method according to any one of [1] to [16] or the manufacturing method according to [17] or [18],

in which microalgae are the microalgae according to [19].

[23] Microalgae as microorganisms of which the identity with base sequences of a partial region corresponding to Chlorococcum sp. RK261 among base sequences encoding a gene region of 18S rRNA is 95.00% to 99.99% or which belong to Chlorococcum sp.,

in which the 18S rRNA gene thereof has sequence identity of at least 99.94% with polynucleotide formed of a base sequence of SEQ ID No: 2.

[24] Microalgae which are Chlorococcum sp. FFG039 strains (accession number of FERM BP-22262) or microalgae having taxonomically the same properties as those of Chlorococcum sp. FFG039 strains.

With use of the method of the present invention, it is possible to easily perform medium replacement while minimizing the influence on a structure of a microalgal biofilm which is a subject to be collected, without using a filter or a centrifugal separator which is complicated and expensive, and of which energy to be input is large. Furthermore, it is possible to perform the collection while minimizing the influence on bottom surface algae at the time of collection using a deposition method, by elevating the water level of a medium, and to suppress decrease in the content of useful substances or decrease in the bottom surface algae as seed algae. In addition, it is possible to reduce the concentration of nutrient source such as nitrogen compound or phosphorus compound by adding a medium, of which the concentration of the nutrient source thereof is small, in the middle of the culturing, and therefore, it is possible to achieve the same effect as that in the medium replacement at lower costs. Furthermore, it is possible to suppress adhesion of a microalgal biofilm on an unexpected position in accordance with the change of the liquid surface, for example, medium replacement, by peeling off the adhesion of the contact point between the wall surface of the culture vessel and the microalgal biofilm, and therefore, it is possible to improve recovery properties.

In addition, it is possible to obtain a high proliferation rate using a medium containing sugar in liquid surface-floating culture, and to obtain high oil content.

### BRIEF DESCRIPTION OF THE DRAWINGS

FIGS. 1A to 1J are schematic views of the present invention. FIG. 1A shows a state in which a suspension liquid of microalgae is put into a culture vessel; FIG. 1B shows a state in which the microalgae sink to the bottom surface of the culture vessel by allowing the microalgae to stand for several seconds to several tens of minutes; FIG. 1C shows a state in which a microalgal biofilm has been formed on the liquid surface by performing culturing and in which the microalgae on the bottom surface have also proliferated at the same time; FIG. 1D shows a state, in which the water surface algae and the bottom surface algae almost come into contact with each other, after a medium is removed; FIG. 1E shows a state in which a medium is added to the culture vessel and culturing is started again; FIG. 1F shows a state in which a first substrate is brought into contact with the microalgal biofilm on the liquid surface, that is, collection is started through transfer; FIG. 1G shows a state in which the substrate to which the microalgal biofilm is attached is taken out of the culture vessel; FIG. 1H shows a state in which the microalgal biofilm on the liquid surface is collected using a second substrate through a deposition method; FIG. 1I shows a state in which a deposited substance is taken out of the culture vessel together with the second substrate; and FIG. 1J shows the culture vessel after the microalgal biofilm on the liquid surface has been collected.

FIG. 2A shows a state which is the same as that of FIG. 1C and FIG. 2B shows a state in which the water depth is made to be deep by adding a medium.

FIG. 3 is a composition of a CSiFF03 medium.

FIG. 4 is a composition of a CSiFF04 medium.

FIG. 5 shows the quantity of dry alga bodies and the oil content when each test is performed. Test Example 1-a shows a result in which a microalgal biofilm on the liquid surface is collected immediately before medium replacement; Test Example 1-b shows a result in a case where replacement of a medium to a CSiFF04 (N-) medium is performed in the middle of culturing which is then further continued. Test Example 1-c shows a result in a case where replacement of the medium to a CSiFF04 medium is performed (that is, the medium is replaced with a fresh medium) in the middle of culturing which is then further continued. Test Example 1-d shows a result in a case where the culturing is continued as it is without performing the medium replacement.

FIG. 6 is a composition of a CSiFF04 (N-) medium.

FIG. 7 is a view in which the results in FIG. 5 are re-calculated as oil content productivity.

FIG. 8A shows water surface-floating algae which have been obtained after a medium has been replaced with a nitrogen compound-removed medium using FFG039 strains as algae; FIG. 8B shows a view in which oil of algal bodies is extracted from the above-described sample using hexane; FIG. 8C is a view in which the above-described oil is analyzed using GC-MS (Gas chromatography mass spectrometer).

FIG. 9 shows the oil content in a case where culturing is continuously performed after decreasing the concentration of the nitrogen compound in the medium by adding distilled water to the culture vessel in the middle of culturing. The ratio of the oil content with respect to dry weight is used.

FIG. 10 shows a view in which the collection of algal bodies was performed through a deposition method without adding distilled water to the culture vessel (upper stage), and a view in which the collection of algal bodies was performed through the deposition method after increasing the water level by adding distilled water to the culture vessel (lower stage).

FIG. 11 shows dry weights of collected algal bodies in a case where microalgae are cultured through liquid surface-floating in media containing glucose at various concentrations.

FIG. 12 shows dry weights of collected algal bodies in a case where FFG039 strains are cultured through liquid surface-floating in media to which various kinds of sugar are added.

FIG. 13 shows dry weights of collected algal bodies in a case where AVFF007 strains are cultured through liquid surface-floating in media to which various kinds of sugar are added.

FIG. 14 is a part of a base sequence (SEQ ID No: 1) of a gene which encodes 18S rRNA of Botryococcus sudeticus AVFF007 strains of microalgae.

FIGS. 15A to 15B show microscopic photographs of Chlorococcum sp. FFG039 strains. FIG. 15A shows a general state and FIG. 15B shows a state in which zoospores are released and proliferated.

FIG. 16 is a gene sequence of the FFG039 strains obtained through 18S rDNA analysis.

FIG. 17 is a genealogical tree of the FFG039 strains.

### DESCRIPTION OF THE PREFERRED EMBODIMENT

Hereinafter, a preferred embodiment of a culture method for microalgae according to the present invention will be described in detail. The numerical range represented by “˜” means a range including numerical values denoted before and after “˜” as a lower limit value and an upper limit value.

[Method of Present Invention]

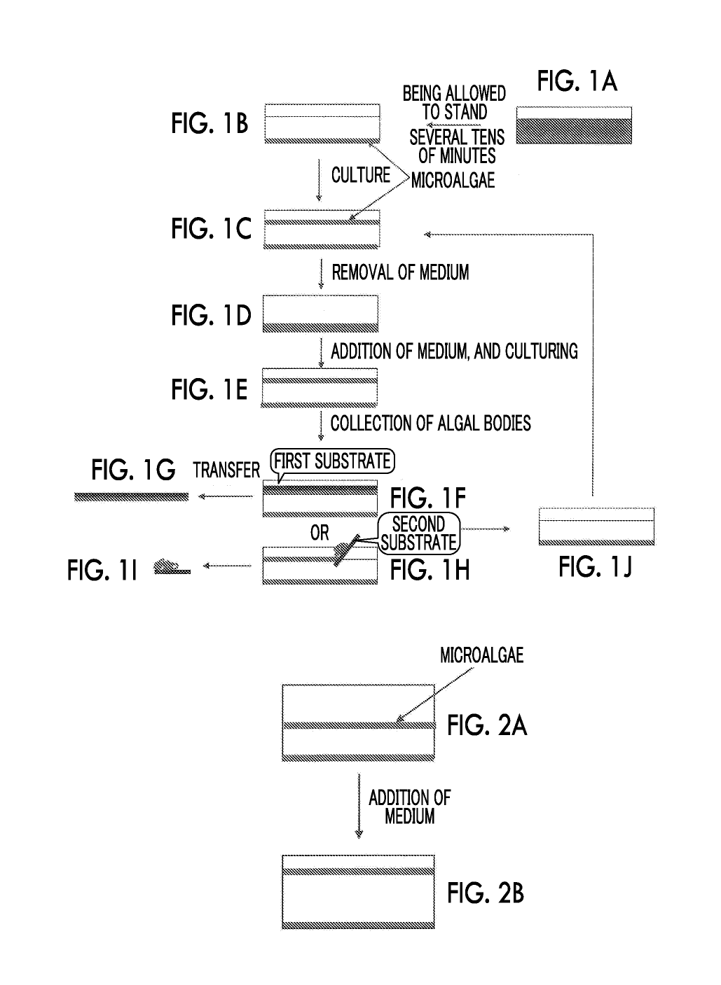

A basic culture method of the present invention is shown in FIGS. 1A to 1J. This schematic view is provided in order to describe the present invention, and therefore, some sections of this drawing are denoted by being simplified.

As shown in FIG. 1A, a suspension liquid of microalgae is prepared and put into a culture vessel. Next, when the culture vessel is in a stationary state, as shown in FIG. 1B, the microalgae sink to the bottom surface of the culture vessel within several seconds to several tens of minutes depending on the types of microalgae. The sinking of microalgae to the bottom surface means that the majority of microalgae sink to the bottom surface, and does not mean a state in which microalgae completely disappear from the top of the liquid surface, the middle of the solution, the side surface of the culture vessel, other surfaces, or in a medium. When the microalgae are cultured in this state for a while, a biofilm formed of the microalgae is formed on the liquid surface as shown in FIG. 1C. The film-like structure changes to a three-dimensional structure in accordance with the progress of the culturing depending on the culture conditions. In addition, as shown in FIG. 1C, the microalgae also exist on the bottom surface of the culture vessel. Moreover, although it is not shown in the drawing, the microalgae also exist on the side surface of the culture vessel or other surfaces. Moreover, although the number of microalgae existing is small, the microalgae also exist in the medium. In the present invention, it is possible to use a medium containing sugar in this process.

In the state of FIG. 1C, at least a part of the medium may be replaced in order to, for example, increase the useful substance content (for example, oil) of the microalgae. For example, the medium can be replaced with a medium of which the concentration or the composition of at least one of a nitrogen compound and a phosphorus compound is different from that of the medium which has been used when starting the culturing before the replacement. For example, it is possible to replace the medium with a medium having a lower concentration. In the present invention, such a method is called medium replacement. The medium replacement is a process shown in FIGS. 1C to 1E. The method of the present invention includes a process in which the concentration of at least one component, such as sugar or nitrogen, contained in a medium is changed, and this process is artificially performed. That is, although the concentration of the component of a medium is generally changed also through metabolism of nutrient components performed by microalgae in culturing of microalgae, the process, which is referred to in the present invention and in which the concentration of at least one component contained in a medium is changed, is not intended to refer to the change due to such metabolism.

When the medium is removed, the microalgal biofilm on the liquid surface approaches the bottom surface in accordance with reduction of the level of the liquid. In a case where the medium is almost completely removed as shown in FIG. 1D, the microalgal biofilms on the liquid surface and on the bottom surface come into contact with each other.

The removal of a medium may mean that the medium may not be completely removed. That is, a part of the medium may remain. In comparison with the amount of liquid in a medium at the time of starting culturing, greater than or equal to 20% of the medium is preferably removed, greater than or equal to 50% of the medium is more preferably removed, and greater than or equal to 80% of the medium is most preferably removed. This is because, with the removal of greater than or equal to 20% of the medium, the efficiency of the replacement of a medium is improved and the amount of useful substances such as oil possessed by microalgae is increased. In the removal of 20% of the medium, the microalgal biofilms on the liquid surface and on the bottom surface do not almost come into contact with each other as shown in FIG. 1C.

Next, with addition of a medium as shown in FIG. 1E, the microalgal biofilm which has been brought into contact with the bottom surface floats on the liquid surface again. At this time, the medium may be added such that the water depth of the medium becomes the water depth before the removal of the medium, or the water depth of the medium may be deeper or shallower than that before the removal of the medium. In addition, a medium having a different composition from that of the medium at the time of starting the culturing is preferably added to the culture vessel, but a medium having the same composition as that of the medium when starting the culturing may be added to the culture vessel. Furthermore, distilled water, ion exchange water, or the like which does not contain nutrients at all may be added to the culture vessel.

Regarding the addition of a medium, in comparison with the amount of liquid in a medium at the time of starting culturing, greater than or equal to 20% of the medium is preferably added to the culture vessel, greater than or equal to 50% of the medium is more preferably added to the culture vessel, and greater than or equal to 80% of the medium is most preferably added to the culture vessel. The upper limit of the amount of the medium to be added is not particularly provided, but less than or equal to 20% of the amount of the medium which can be introduced into the culture vessel is preferable, less than or equal to 50% of the amount of the medium thereof is more preferable, and less than or equal to 90% of the amount of the medium thereof is most preferable.

The medium replacement is preferably performed from a region between the liquid surface and the bottom surface. This is performed in order to prevent the structure of the microalgal biofilm on the liquid surface from being significantly destroyed due to the operation of the medium replacement and to prevent a large amount of the microalgal biofilm from being removed due to the operation of the medium replacement. Such purposes can be achieved by performing the medium replacement through, for example, installation of a pipe for the medium replacement on the side surface or the like of the culture vessel or through installation of a mobile pipe. In addition, a part of the structure of the microalgal biofilm which has been formed on the liquid surface can be destroyed and a suction port for aspirating the medium can be inserted through the destroyed biofilm to pump the medium from the region between the liquid surface and the bottom surface. Accordingly, although only a part of the biofilm structure is destroyed, the majority thereof is not destroyed, which is preferable. Performing of the removal of the medium from the region between the liquid surface and the bottom surface means performing the medium replacement on a region in which there is almost no microalga between the microalgal biofilms on the liquid surface and on the bottom surface. This is because it seems that there is almost no microalga in this region at least according to visual observation, except for zoospores or the like. In addition, in a case where the microalgal biofilms on the liquid surface and on the bottom surface come into contact with each other, it is impossible to add a medium by inserting a pipe or the like between the microalgal biofilms on the liquid surface and on the bottom surface. Therefore, it is desirable that a medium is supplied through an injection port which is positioned higher than the microalgal biofilm on the liquid surface.

In addition, as shown in the processes of FIGS. 2A and 2B, it is possible to decrease the concentration of a nitrogen compound or a phosphorus compound in a medium by adding a medium having a lower concentration of a nitrogen compound or a phosphorus compound than that of the medium at the time of starting culturing, without removing the medium. FIG. 2A corresponds to FIG. 1C and FIG. 2B corresponds to FIG. 1E. In addition, in the present invention, such a method is also regarded to be called medium replacement.

In many cases, in a site in which microalgae on the liquid surface and the wall surface of a culture vessel come into contact with each other, a microalgal biofilm adhere to the wall surface thereof. In this case, there are problems in that the microalgal biofilm adhere to an unexpected site in accordance with fluctuation of the liquid surface due to medium replacement and the structure of the microalgal biofilm is destroyed. In order to solve such problems, it is possible to perform an operation of peeling off the adhesion site between a microalgal biofilm on the liquid surface and the wall surface of a culture vessel using a metallic spatula. Accordingly, the microalgal biofilm can be moved in accordance with fluctuation of the liquid surface while maintaining the form thereof. In some cases, the amount of liquid of a medium is gradually decreased through evaporation outer layer in accordance with the progress of the culturing. In this case, it is possible to peel off the adhesion site between the biofilm and the wall surface after adding an amount of liquid corresponding to the amount of liquid which has been lost. The peeling method is not particularly limited as long as it is possible to achieve the purposes. It is possible to use a stick such as a metallic spatula, a film, or the like. In addition, it is also possible to peel off the adhesion site therebetween using a wave or an ultrasonic wave of the liquid surface without using an instrument.

After performing such processing, the culturing is continued for a while. Microalgae accumulate useful substances such as oil through this process.

Thereafter, microalgal biomass on the liquid surface is collected. The biomass can be collected through a transferring method using a first substrate as shown in FIG. 1F or through a deposition method using a second substrate as shown in FIG. 1H. The states in which the substrates are taken out of the culture vessel are respectively states of of FIGS. 1G and 1I. After collecting microalgae on the substrates, a product is obtained through a necessary process. In the schematic view, the substrates to which microalgae adhere are moved outside the culture vessel. However, the collected substance may be collected from the substrates within the culture vessel.

The state after the biofilm on the liquid surface is collected is FIG. 1J. Here, microalgae remain on the bottom surface of the culture vessel. The culturing can be repeated many times using these microalgae. Even at this time, medium replacement may be performed. However, the medium may be replaced with a medium containing many nitrogen compounds or phosphorus compounds.

[Medium Containing No Nitrogen Compound or Medium in which Nitrogen Compound is Reduced]

According to an example of the embodiment of the present invention, a suspension liquid or a dispersion liquid which contains microalgae obtained through a purification process is prepared by dispersing the microalgae in a liquid medium including an artificial medium; a biofilm of the microalgae is formed on the liquid surface of the liquid medium by culturing the prepared suspension liquid or dispersion liquid in a culture vessel; and medium replacement is performed; and then, the culturing is continuously performed.

In the present invention, a medium which contains no nitrogen compound such as a nitrate or in which a nitrogen compound is reduced (in some cases, such a medium is represented by “N-”) can be used as previously stated. Examples of the medium containing no nitrogen compound which is used for culturing microalgae include a CSiFF04 (N-) medium shown in FIG. 6 or an IMK(N-) medium. The composition of the medium is not limited thereto as long as no nitrogen compound is contained therein.

Containing no nitrogen compound means that a nitrogen compound represented by a nitrate body (in more specific, potassium nitrate or the like) is not contained (a nitrogen compound is not detected or is less than 40 μg/mL as the amount of nitrate nitrogen) at a point in time when starting culturing (initial concentration). The medium in which the nitrogen compound is reduced means a medium of which the concentration of the nitrogen compound is three-fourths or less of the concentration of the nitrogen compound in the medium at the time of starting culturing, is preferably two-thirds or less of the concentration of the nitrogen compound in the medium at the time of starting culturing, and more preferably one half or less of the nitrogen compound in the medium at the time of starting culturing. Such a medium can be prepared by diluting a medium having a standard composition using water or an adequate buffer solution or by containing no nitrogen compound or phosphorus compound at the time of preparing a medium.

Similarly, in some cases, a medium which contains no phosphorus compound or in which a phosphorus compound is reduced can be used in the present invention.

[Sugar]

According to an example of the embodiment of the present invention, a suspension liquid or a dispersion liquid which contains microalgae obtained through a purification process is prepared by dispersing the microalgae in a liquid medium (including an artificial medium) containing sugar which can be assimilated by the microalgae; and a biofilm of the microalgae is formed on the liquid surface of the liquid medium by culturing the prepared suspension liquid or dispersion liquid in a culture vessel.

With use of the medium containing sugar, in some cases, it is possible to suitably improve the proliferation rate compared to a case in which light and carbon dioxide are used. In addition, the oil content tends to increase.

Sugar which can be assimilated by microalgae and can be used in the present invention includes at least monosaccharide, disaccharide, trisaccharide, or polysaccharide. As monosaccharide, it is possible to use any well-known monosaccharide such as galactose, mannose, talose, ribose, xylose, arabinose, erythrose, threose, glyceraldehyde, fructose, xylulose, or erythrulose. As disaccharide, it is possible to use any well-known disaccharide such as trehalose, kojibiose, nigerose, maltose, or isomaltose. In addition, either of triose, tetrose, pentose, hexose, or heptose can be used. As polysaccharide, it is possible to use starch, amylose, glycogen, cellulose, or the like. In addition, as oligosaccharide, it is possible to use galactooligosaccharide, deoxyribose, glucuronic acid, glucosamine, glycerin, xylitol, or the like.

As the concentration of sugar in a medium, greater than or equal to 0.1 μg/mL is preferable, greater than or equal to 0.1 mg/mL is more preferable, and greater than or equal to 1 mg/mL is most preferable. If the concentration of sugar thereof is greater than or equal to 0.1 μg/mL, it is possible to suitably improve the proliferation rate of microalgae, which is preferable. In addition, although the upper limit thereof is not particularly provided, the concentration thereof is preferably lower than or equal to the degree of solubility, more preferably half or less of the degree of solubility, and still more preferably one-tenth of the degree of solubility. More specifically, in a case where glucose is used as sugar, the amount of glucose can be set to be less than or equal to 30 mg/mL, preferably set to be less than or equal to 10 mg/mL, and more preferably set to be less than or equal to 5 mg/mL. The concentration of sugar is a concentration (initial concentration) immediately before starting culturing, and in many cases, the concentration of sugar in a medium is continuously changed.

As sugar, a single kind of sugar may be used or two or more kinds of sugar may be used.

In the present invention, it is possible to use both of light and sugar and to perform culturing only using sugar without light.

In the case of using sugar, it is preferable to use a closed-type culture vessel in order to improve the proliferation rate of bacteria other than microalgae, compared to the case of using light and carbon dioxide. That is, in the case of using the closed-type culture vessel, bacteria in the outside air are mixed in and consume sugar in a medium.

In addition, culturing may be performed in combination of light and sugar; light, carbon dioxide, and sugar; and carbon dioxide and sugar.

In addition, in the present invention, it is possible to use sugar generated through metabolism or the like of microorganisms. Furthermore, it is possible to use sugar generated through metabolism of microorganisms in the outside of a culture vessel. Moreover, it is also possible to use sugar generated through metabolism or the like of microorganisms by simultaneously culturing microalgae and microorganisms.

[Microalgae]

Microalgae of the present invention indicate minute algae of which the individual existence cannot be identified with the naked eye. Microalgae are not particularly limited as long as the microalgae have an ability of forming a biofilm on the liquid surface, and either prokaryote or eukaryote may be used.

The microalgae are not particularly limited, and any microalgae can be appropriately selected in accordance with the purpose. Examples thereof include the division Cyanophyta, the division Glaucophyta, the division Rhodophyta, the division Chlorophyta, the division Cryptophyta, the division Haptophyta, the division Heterokontophyta, the division Dinophyta, the division Euglenophyta, and the division Chlorarachniophyta. These may be used alone or in a combination of two or more thereof. Among these, as the above-described microalgae, the division Chlorophyta is preferable, and green algae are more preferable. As the microalgae, the genus Haematococcus (Haematococcus sp.), the genus Chlamydomonas (Chlamydomonas sp.), the genus Chlorococcum (Chlorococcum sp.), and the genus Botryococcus (Botryococcus sp.) are more preferable in terms of production of biomass.

The above-described method of obtaining microalgae is not particularly limited, and any method can be appropriately selected in accordance with the purpose. Examples thereof include a method of collecting microalgae in nature, a method of using a commercially available product, and a method of obtaining microalgae from a culture collection or a depositary institution. Microalgae used in the present invention are preferably microalgae obtained through a purification process. The purification process is a process which is performed for the purpose of making microalgae be a single type, and it is unnecessary to make microalgae be completely a single type.

In the present invention, among the above-described microalgae, microalgae which can produce useful substances are preferable. Particularly, microalgae which produce an intermediate or a final product for a pharmaceutical product, a cosmetic, or a health food product; a raw material used in synthetic chemistry; a hydrocarbon compound or triglyceride; an oily substance such as fatty acid compound; gas such as hydrogen; and the like are preferable. In the present invention, in some cases, these are called products. Furthermore, in the present invention, it is preferable to use microalgae which satisfy either of good culturing on the liquid surface and good recovery from the liquid surface; possession of a high proliferation rate; possession of a high oil content ratio; generation of little odor at least during culturing; or no generation of poisonous substances being confirmed.

[Biofilm]

The biofilm in the present invention refers to a structure of microalgae (microalgae aggregation or microalgae film; biofilm) which is adhered to the surface of rock or the like. Besides these, in the present invention, a film-like structure or a three-dimensional structure, which is formed of microalgae existing on the surface such as the liquid surface having fluidity, is also called a biofilm. A biofilm in nature also contains debris or pieces of plants besides target microalgae. The biofilm of the present invention may contain these as long as the biofilm is a sample which has been obtained through a purification process. However, ideally, it is more preferable that the biofilm is formed of only the microalgae according to the present invention and a substance such as an intracellular matrix which is secreted during the proliferation of the microalgae. In addition, microalgae on the bottom surface can also be called a biofilm as long as the microalgae form a film-like structure.

In addition, it is preferable that the biofilm is configured such that individual microalgae are adhered to each other directly or via a substance (for example, polysaccharides) such as the intercellular matrix.

In the present invention, it is necessary to use microalgae capable of forming a biofilm on the liquid surface. Preferred examples of such microalgae include Botryococcus sudeticus or the genus Chlorococcum. More specific examples thereof include Botryococcus sudeticus AVFF007 strains (hereinafter, simply referred to as AVFF007 strains) or FFG039 strains. As a result of analyzing a gene sequence which encodes 18S rRNA, the FFG039 strains are identified as Chlorococcum sp.

[AVFF007 Strains]

The AVFF007 strains as microalgae used in Examples of the present specification are internationally deposited to the Patent Organism Depository of the National Institute of Advanced Industrial Science and Technology (Central 6, 1-1, Higashi 1-chome, Tsukuba-shi, Ibaraki-ken, Japan) dated Sep. 28, 2011 with a accession number of FERM BP-11420 under the Budapest Treaty, by FUJIFILM Corporation (26-30, Nishiazabu 2-chome, Minato-ku, Tokyo, Japan). The work of the Patent Organism Depository of the National Institute of Advanced Industrial Science and Technology was handed over to the Patent Organism Depositary of the National Institute of Technology and Evaluation (Room No. 120, 2-5-8 Kazusakamatari, Kisarazu-shi, Chiba-ken, Japan) from Apr. 1, 2012.

The AVFF007 strains are novel strains of freshwater microalgae which have been isolated from a freshwater pond in Kyoto in Japan by the present inventors. A part (SEQ ID No: 1, FIG. 14) of a base sequence of the 18S rRNA gene was analyzed using BLAST based on data of the National Center for Biotechnology Information (NCBI). As a result, the part of the base sequence thereof was identified as microalgae which were closely related to Botryococcus sp. UTEX2629 (Botryococcus sudeticus) strains (homologous to a 1109 base on the AVFF007 strain side among a 1118 base on the UTEX2629 strain side). The AVFF007 strains are microalgae which are also closely related to Characiopodium sp. Mary 9/21 T-3w, and there is also a possibility that the name of the AVFF007 strains may be changed to the genus Characiopodium. In this case, the name of the AVFF007 strains is regarded to be changed thereto. In addition, in a case where the name of the AVFF007 strains is changed to a name other than the genus Characiopodium, the name of the AVFF007 strains in the present invention is also regarded as to be changed thereto.

In the present invention, it is possible to use strains having taxonomically the same properties as those of the AVFF007 strains. The taxonomic properties of the AVFF007 strains are shown below.

Taxonomic Properties of AVFF007 Strains

### 1. Morphological Properties

The AVFF007 strains have a green circular shape. The AVFF007 strains have floating properties, and therefore, can proliferate on the liquid surface and the bottom surface. The size of an AVFF007 strain is 4 μm to 30 μm (the size of AVFF007 strain on the liquid surface is comparatively large, but the size of AVFF007 strain on the bottom surface is comparatively small). The AVFF007 strains proliferate on the liquid surface and form a film-like structure. Bubbles are formed on the liquid surface in accordance with the proliferation and overlap each other to form a three-dimensional structure on the liquid surface. In addition, oil is produced.

### 2. Culturing Properties (Culture Method)

(1) Medium: CSiFF04 (which is obtained by improving a CSi medium and of which the composition is shown in FIG. 4) of which the pH is adjusted to 6.0 using NaOH or HCl. The medium can be sterilized at 121° C. for 10 minutes.
(2) Culture temperature: a favorable temperature is 23° C. and culturing can be performed at less than or equal to 37° C.
(3) The culture period (the period until the culturing generally reaches a stationary phase) depends on the quantity of algal bodies which has been initially used, and is 2 weeks to 1 month. In general, culturing can be performed at 1×10⁵ cells/mL.
(4) Culture method: aerobic culture and stationary culture are suitable.
(5) Light-requiring properties: Necessary. Intensity of light: 4000 lux to 15000 lux. Bright and dark cycle: 12 hours for time for a bright period and 12 hours for time for a dark period. During subculture, it is possible to perform the culturing at 4000 lux.

The AVFF007 strains can be stored through the subculture in accordance with the above-described culturing properties (culture method). The subculture can be performed by collecting microalgae which float on the liquid surface and performing dispersion such as pipetting, and then, dispersing the microalgae in a fresh medium. Immediately after the subculture, the microalgae are sunk on the bottom surface of a culture vessel, and start to form a biofilm on the liquid surface after about one week. Proliferation can be performed even if the microalgae exist on the liquid surface immediately after the subculture. The cycle for the subculture is about one month. Subculture is performed when the microalgae exhibit yellow color.

As strains having the taxonomically same properties as those of the AVFF007 strains, microalgae are included of which the 18S rRNA gene has sequence identity of at least 95.0%, preferably 98.0%, more preferably 99.0%, still more preferably 99.5%, and most preferably 99.9% with polynucleotide formed of a base sequence of SEQ ID No: 1.

The sequence identity mentioned regarding the base sequence in the present invention means a percentage of the number of common bases which are coincident with each other between two arrayed bases within a region in a case where the two bases are arrayed in an optimal mode. That is, the identity can be calculated through an equation of identity=(number of coincident bases/total number of bases)×100 and can be calculated using an algorithm which is commercially available or open to the public. Search and analysis with respect to the identity of the base sequence can be performed using an algorithm or a program well known for those skilled in the art. In a case of using a program, those skilled in the art can appropriately set the parameter. Alternately, a default parameter of each program may be used. Specific techniques for the analysis method thereof are also well known to those skilled in the art.

[FFG039 Strains]

The FFG039 strains as microalgae used in Examples of the present specification are collected by the present inventors from Nara Prefecture in Japan. The FFG039 strains have good proliferating properties and are excellent in oil productivity compared to the AVFF007 strains. In addition, the FFG039 strains have characteristics in that the structure of the biofilm is hardly destroyed and it is easy to collect the FFG039 strains. The FFG039 strains are Chlorococcum sp. As a result of analysis of the gene sequence of 18S rRNA, the FFG039 strains are species closely related to RK261 strains (Chlorococcum sp. RK261) of the genus Chlorococcum as microalgae. In the present invention, newly isolated microalgae are named Chlorococcum sp. FFG039. It is more preferable that the identity with base sequences of a partial region corresponding to RK261 of the genus Chlorococcum among base sequences encoding a gene region of the microalgae according to the present invention is 95.00% to 99.99%. The “partial region” referred to herein means a region having greater than or equal to 1000 base sequences. When testing the identity, use of every base sequence results in the highest reliability for the test of the identity. However, determining every base sequence is technically and financially difficult except for an extremely small number of species of organisms. In addition, only a specific portion (specifically, the vicinity of base sequences corresponding to base sequences of the Chlorococcum sp. FFG039 strains (hereinafter, also simply referred to as FFG039 strains) which are set as a comparison target to be described below) of the base sequences of the RK261 strains of the genus Chlorococcum has not been disclosed. Furthermore, in general, it is considered that there can be attribution if about 1000 base sequences are read. From the above, the identity has been tested through the comparison with, the base sequences of a “partial region” in the present invention, and it is considered that the reliability thereof is sufficiently high. The Japanese name of Chlorococcum is based on the Japanese name disclosed in Freshwater algae, written by Takaaki YAMAGISHI, UCHIDA ROKAKUHO PUBLISHING CO., LTD.

The FFG039 strains as microalgae used in Examples of the present specification are internationally deposited to the Patent Organism Depositary of the National Institute of Technology and Evaluation (Room No. 120, 2-5-8 Kazusakamatari, Kisarazu-shi, Chiba-ken, Japan) dated Feb. 6, 2014 with a accession number of FERM BP-22262 under the Budapest Treaty, by FUJIFILM Corporation (26-30, Nishiazabu 2-chome, Minato-ku, Tokyo, Japan).

The FFG039 strains are novel strains of freshwater microalgae which have been isolated from a pond in Kyoto in Japan by the present inventors and belong to the genus Chlorococcum.

Hereinafter, the method of isolating the microalgae (hereinafter, also referred to as purification) and circumstances in which it has been determined that the FFG039 strains of the microalgae are novel strains will be described.

[Purification of FFG039 Strains of Microalgae]

Natural fresh water was collected from a pond in Nara Prefecture by putting it into a 5 mL tube for homogenizing (TM-655S, Tomy Seiko Co., Ltd.). 100 μL of the collected natural fresh water was added to a 24 hole plate (microorganism culture plate 1-8355-02, As One Corporation) into which 1.9 mL of a medium, in which a CSiFF04 medium shown in FIG. 4 was put. The plate was placed on a plant bioshelf for tissue culture (AV152261-12-2, Ikeda Scientific Co., Ltd.) and was cultured at 23° C. under continuous irradiation with 4000 lux light. After approximately one month, a green aggregation was generated in the wells of the 24 hole plate. The aggregation was observed using an optical microscope and it was confirmed that there were a large number of microorganisms.

1 g of agarose (Invitrogen, UltraPure™ Agarose) was weighed out, and 200 mL of a CSiFF04 medium was put into a 500 mL conical flask. The CSiFF04 medium was subjected to an autoclave treatment for 10 minutes at 121° C., and approximately 20 mL of the CSiFF04 medium at a time was added to an Azunol Petri dish (1-8549-04, As One Corporation) in a clean bench before being cooled and hardened to produce agarose gel.

The solution containing the microalgae in the 24 hole plate was diluted, and the solution was made to adhere to a loop portion of a disposable stick (1-4633-12, As One Corporation) and was applied to the prepared agarose gel to prepare a Petri dish in which the microalgae were applied to the agarose gel.

The Petri dish was placed on a plant bioshelf for tissue culture and was cultured at 23° C. under continuous irradiation with 4000 lux light. After approximately 2 weeks, a green colony appeared on the agarose gel. The colony was adhered to a distal end of a sterilized bamboo skewer (1-5980-01, As One Corporation), and then, was suspended in the wells of the 24 hole plate into each of which 2 mL of the CSiFF04 medium was put. The 24 hole plate containing microalgae prepared in this manner was placed on a plant bioshelf for tissue culture and was cultured at 23° C. under continuous irradiation with 4000 lux light. After approximately 2 weeks, the aqueous solution in the wells exhibited a green color. A small amount of solution was collected from all of the wells, the microalgae were observed using an optical microscope, and it was concluded that purification had been performed in wells, in which it was considered that there was only a single microalga.

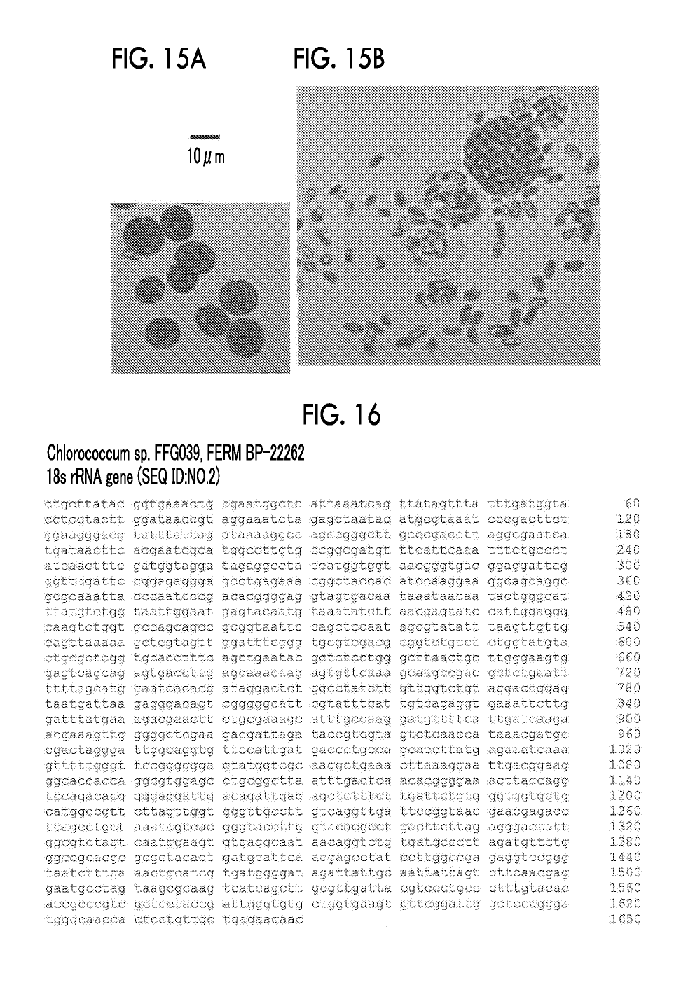

In addition, a microphotograph of FFG039 strains at magnification of 40 is shown in FIGS. 15A and 15B. FIG. 15A shows an ordinary state and FIG. 15 B shows that the FFG039 strains proliferate by releasing a large number of zoospores.

[Morphological Properties]

All microalgae sink to the bottom surface if the microalgae are left for a while after performing a dispersion treatment.

If the microalgae are cultured for a while, microalgae floating on the liquid surface appear. Accordingly, microalgae are divided into microalgae having sunk to the bottom surface and microalgae floating on the liquid surface. If further culturing is performed continuously, a film-like structure appears on the liquid surface. If the culturing is performed further, a three-dimensional structure appears.

All the microalgae on the liquid surface and on the bottom surface have a spherical shape and have different size distributions.

The microalgae have cohesiveness and form a large colony.

The microalgae are green and the color thereof turns to yellow in accordance with the progress of the culturing.

There is little odor in collected substances and during the culturing.

[Culturing Properties]

During the cell proliferation, the cells proliferate through zoospores. A large number of zoospores are generated from one cell.

It is possible to perform photoautotrophic culture through photosynthesis.

Nitrogen, phosphorus, potassium, calcium, magnesium, sulfur, manganese, and iron are essential for proliferation. In addition, inclusion of zinc, cobalt, molybdenum, and boron makes the proliferation favorable. Addition of vitamins also promotes the proliferation.

[Physiological Properties]

Oil accumulates in an algal body at a maximum close to 40 wt % in terms of dry weight proportion.

In the oil, hydrocarbon compounds and fatty acids accumulate. The fatty acids produce palmitic acid, palmitoleic acid, oleic acid, vaccenic acid, linoleic acid, linolenic acid, and the like, and particularly, palmitic acid and oleic acid are main components. The hydrocarbon compounds produce decane, heptadecane, and the like.

FFG039 strains dyed with Nile Red were observed by a fluorescence microscope. Then, it was confirmed that there was oil which was colored with the Nile Red as a bright fluorescent light-emitting region in algal bodies in a fluorescent visual field. The oil can accumulate in a comparatively wider region within an alga body cell.

Identification of the FFG039 strains was further performed through the following method.

(Identification of FFG039 Strains of Microalgae)

The culture method of the FFG039 strains was as follows. 50 mL of a CSiFF04 medium was introduced into a conical flask with a 100 mL capacity, 0.5 mL of an FFG039 strain solution at a concentration of 1000×10⁴ cells/mL was added thereto, and shaking culture was performed under light irradiation for 14 days at 25° C.

In order to obtain a dry powder of the FFG039 strains, a centrifugal operation was performed on 40 mL of the medium containing the FFG039 strains obtained as described above using a centrifuge (MX-300 (Tomy Seiko Co., Ltd.) for 10 minutes at a centrifugal force of 6000×g below 4° C. After removing a supernatant, the solid body was frozen together with the container using liquid nitrogen. Then, the total quantity of frozen solid body was transferred to a mortar which was chilled in advance using liquid nitrogen, and was ground using a pestle which was chilled in advance using liquid nitrogen.

The extraction of DNA from the microalgae was performed using DNeasy Plant Mini Kit (manufactured by Qiagen) according to the described manual. The purity and the amount of the extracted DNA were measured using e-spect (manufactured by Malcom Co., Ltd.). It was confirmed that the extracted DNA achieved the index of a purification degree which is A₂₆₀ₙₘ/A₂₈₀ₙₘ=1.8 or greater, and about 5 ng/μL of DNA was taken.

There was no problem in the purification degree of the extracted DNA, and thus, a sample for PCR was prepared by diluting the DNA 10⁴ times in ultrapure water. An 18S rRNA gene region (rDNA region) was used as the sample for PCR. A cycle including 10 seconds at 98° C., 50 seconds at 60° C., and 10 seconds at 72° C. was performed 30 times for the PCR using a GeneAmp PCR System 9700 (manufactured by Applied Biosystems). An enzyme used herein was Prime Star Max (manufactured by Takara Bio Inc.). It was confirmed through 1% agarose electrophoresis that the obtained PCR product was a single band.

The purification of the PCR product was performed using a PCR purification kit (manufactured by Qiagen). The method was carried out through the method described in the manual. In order to check the purification degree and whether the PCR reaction was sufficiently performed, the purity and the amount of the DNA were measured using e-spect. It was determined that there was no problem since the measured purification degree was A₂₆₀ₙₘ/A₂₈₀ₙₘ=1.8 or greater.

Next, the purified substance was used as a template and a cycle sequence was performed using a BigDye Terminator v3.1 Cycle Sequencing kit (manufactured by Applied Biosystems). The manual was referred to for the condition. The base sequence of the obtained reactant was decoded using ABI PRISM 3100-Avant Genetic Analyzer (manufactured by Applied Biosystems).

Identity analysis was performed using a basic local alignment search tool (BLAST). The method thereof was as follows. BLAST searching for the above-described sequence was conducted on the whole base sequence information in the data of the National Center for Biotechnology Information (NCBI). A species of an organism having the highest identity was regarded as a closely related species of the FFG039 strains. Only the base sequence (1650 base, SEQ ID No: 1) which was set as a comparison target is shown in FIG. 16. Specifically, several bases at both the ends of the decoded base sequence were not set as a comparison target for the BLAST analysis, and thus are not shown in FIG. 16. The upper left of the base sequence shown in FIG. 16 is a 5′-terminal and the lower right thereof is a 3′-terminal.

As a result of the identity analysis, the above-described sequence had the identity (that is, 99.94% identity) to a 1649 base on the FFG039 strain side among a Chlorococcum sp. RK261 strain side and 1650 bases on the Chlorococcum sp. RK261 strain side. Accordingly, the FFG039 strains were classified as microalgae closely related to the Chlorococcum sp. RK261 strains.

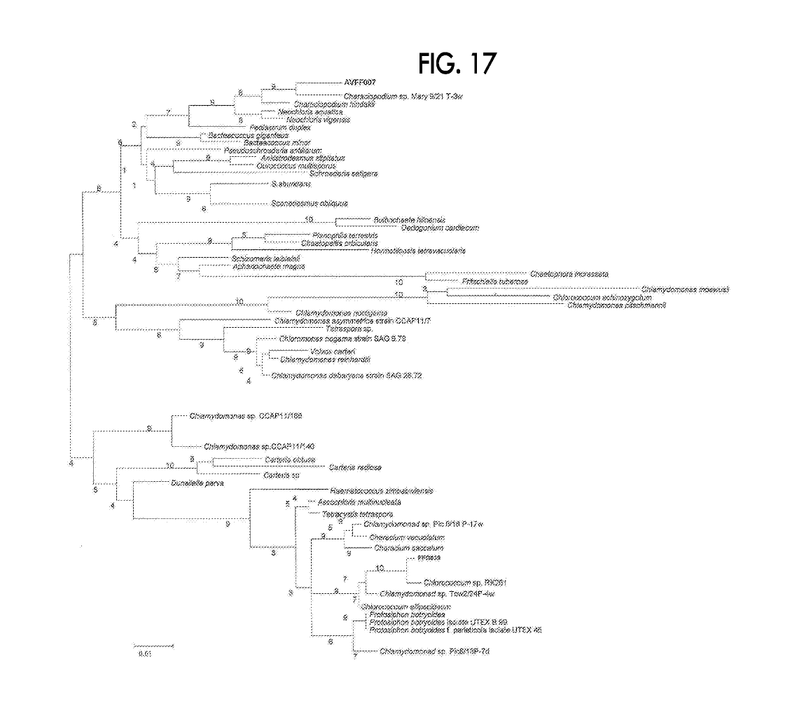

The systemic diagram obtained from the results of the above-described analysis is shown in FIG. 17. In a case where the name of the Chlorococcum is changed, similarly, it is regarded that the name of the FFG039 strains is also changed in the present invention.

In the present invention, it is possible to use strains having taxonomically the same properties as those of the FFG039 strains. The taxonomic properties of the FFG039 strains are shown below.

Taxonomic Properties of FFG039 Strains

### 1. Morphological Properties

The AVFF007 strains have a circular shape. When stationary culture is performed, a film-like structure is formed on the liquid surface. Oil is produced.

### 2. Culturing Properties (Culture Method)

(1) Medium: a CSiFF04 medium or a CSi improved medium (150 mg/L of Ca(NO₃)₂.4H₂O, 100 mg/L of KNO₃, 28.4 mg/L of K₂HPO₄, 22.2 mg/L of KH₂PO₄, 40 mg/L of MgSO₄.7H₂O, 588 ug/L of FeCl₃.6H₂O, 108 ug/L of MnCl₂.4H₂O, 66 ug/L of ZnSO₄.7H₂O, 12 ug/L of CoCl₂.6H₂O, 7.5 ug/L of Na₂MoO₄.2H₂O, 3 mg/L of Na₂EDTA.2H₂O, 0.1 ug/L of vitamin B12, 0.1 ug/L of Biotin, 10 ug/L of thiamine.HCl, pH 7.0)
(2) Culture temperature: culturing can be performed at 15° C. to 25° C.
(3) Culture period: for 2 to 4 weeks
(4) Culture method: stationary culture is suitable.
(5) Light-requiring properties: Necessary. Intensity of light: 4000 lux to 15000 lux. Bright and dark cycle: 12 hours for time for bright period and 12 hours for time for dark period.

As microalgae having the taxonomically same properties as those of the FFG039 strains, microalgae which belong to the genus Chlorococcum sp. and of which the 18S rRNA gene has sequence identity of at least 99.94% with polynucleotide formed of the base sequence of SEQ ID No: 2 are included.

[Existence State of Microalgae]

In the present invention, microalgae which can form a biofilm on the liquid surface and have at least one characteristic selected from the group consisting of the following (1) to (8) when being cultured in a medium within a culture vessel may be used. With the use of such microalgae, it is possible to expect to perform the present invention, in which it is easier to perform operations such as medium replacement, recovery of a biofilm, and restart of culturing, and the costs are further reduced.

(1) The sum of the quantity of algal bodies of microalgae existing on the liquid surface and in a region from 1 cm below the liquid surface to the liquid surface, and the quantity of algal bodies of microalgae on the bottom surface of a culture vessel is greater than or equal to 10 times, preferably 20 times, and more preferably 30 times the quantity of algal bodies existing in the other region within the culture vessel. The other region within the culture vessel indicates a region excluding the top of the liquid surface, the vicinity of the liquid surface, that is, the region from 1 cm below the liquid surface to the liquid surface, and the bottom surface. In some cases, microalgae adhere on the surfaces of various structures, such as a sensor monitoring the culture, which are installed on the side surface of the culture vessel and in the culture vessel. However, such microalgae may not be included in any of the regions. The quantity of algal bodies can be represented as the weight of algal bodies per area of the bottom surface of the culture vessel.
(2) The specific gravity of microalgae on the liquid surface is smaller than that of microalgae on the bottom surface of the culture vessel. The specific gravity of microalgae can be obtained through a well-known method, for example, a concentration gradient method. The specific gravity of microalgae on the liquid surface when the specific gravity of microalgae on the bottom surface is set to 1 depends on the types of microalgae, and is for example, less than or equal to 0.99, preferably less than or equal to 0.98, and more preferably less than or equal to 0.96. The lower limit value is not particularly limited, but in any case, the upper limit value is, for example, greater than or equal to 0.75, preferably greater than or equal to 0.77, and more preferably greater than or equal to 0.79.
(3) The specific gravity of microalgae on the liquid surface is greater than that of water.
(4) The oil content of microalgae on the liquid surface is higher than that of microalgae on the bottom surface. The oil content of microalgae on the liquid surface when the oil content of microalgae on the bottom surface is set to 1 is, for example, greater than or equal to 1.1, preferably greater than or equal to 1.2, and more preferably greater than or equal to 1.3. The upper limit value is not particularly limited, but in any case, the lower limit value is, for example, less than or equal to 3.0, preferably less than or equal to 2.5, and more preferably less than or equal to 2.0.
(5) The size (diameter) of microalgae on the liquid surface is larger than that of microalgae on the bottom surface. The size of microalgae can be obtained through a well-known method. The size of microalgae on the liquid surface when the size of microalgae on the bottom surface is set to 1 is, for example, greater than or equal to 1.5, preferably greater than or equal to 1.8, and more preferably greater than or equal to 2.0. The upper limit value is not particularly limited, but in any case, the lower limit value is, for example, less than or equal to 4.0, preferably less than or equal to 3.5, and more preferably less than or equal to 3.0.
(6) A formed biofilm includes a film-like outer layer and an inner layer which has a plurality of bubble-like structures, and the outer layer is thicker than the inner layer. The thickness of the layers can be obtained through a well-known method. The thickness of the outer layer when the thickness of the inner layer is set to 1 is, for example, greater than or equal to 2.0, preferably greater than or equal to 3.0, and more preferably greater than or equal to 5.0. The upper limit value is not particularly limited, but in any case, the lower limit value is, for example, less than or equal to 18.0, preferably less than or equal to 14.0, and more preferably less than or equal to 10.0. In some cases, a formed biofilm only includes an outer layer. Accordingly, a formed biofilm having any one of the film-like outer layer and the inner layer which has a plurality of bubble-like structures can be regarded as one characteristic which microalgae of the present invention has.
(7) A part of a formed biofilm has a pleat-like structure in a medium.
(8) In a case where microalgae obtained by collecting a biofilm which has been formed and subjecting the collected biofilm to suspension treatment are seeded on the liquid surface of a medium, the microalgae can be deposited in the medium. In general, a biofilm formed on the liquid surface can be collected, and then, can be carefully applied to the liquid surface as it is without being subjected to suspension treatment so as to be made float on the liquid surface. However, in many cases, through the suspension treatment, it is difficult to make the microalgae float on the liquid surface and the microalgae become deposited.

Microalgae which can form a biofilm on the liquid surface referred to in the present invention and have at least one characteristic selected from the group consisting of the above (1) to (8) when being cultured in a medium within a culture vessel refer to an aggregation of microalgae which can be distinguished from an aggregation of other algae having at least one characteristic selected from the group consisting of the above (1) to (8) and can proliferate while having at least one characteristic thereof. The above-described (3), (4), and (5) can be determined by obtaining an average specific gravity, oil content, or size of microalgae which become a subject.

[Floating Culture]

In the present invention, the culturing of microalgae in a medium in a state of being dispersed is referred to as floating culture. In the present invention, the culturing on the liquid surface is not referred to as floating culture. The floating culture is not performed in a primary culture process, but can be used in accordance with the purpose in a pre-culture process.

[Stationary Culture]

In the primary culture process in the present invention, it is preferable to perform stationary culture. The stationary culture is referred to as a culture method in which a medium or the like is not moved intentionally during culturing.

[Liquid Surface-Floating Culture]

In the present invention, a culture method for culturing microalgae on the liquid surface is referred to as liquid surface-floating culture. Even if microalgae exist on the bottom surface of a culture vessel, on a side surface thereof, on other surfaces, in the middle of a medium, or the like at the same time, the culture method is also referred to as liquid surface-floating culture in a case where the main purpose of the culture method is to perform culturing on the liquid surface. In addition, a large amount of bubble exists on the liquid surface together with a biofilm, and therefore, in some cases, the position of the liquid surface is not always clear. In addition, in some cases, the biofilm is slightly sunk under the liquid surface due to its own weight. Such a case is included as well as a complete liquid surface when the top of the liquid surface is mentioned in the present invention. However, a culture method in which microalgae are cultured in either or only both of the middle of a solution or the bottom surface of a culture vessel is not included in the liquid surface-floating culture.

The liquid surface in the present invention refers to a typical liquid surface of a liquid medium to be described below, and in general, refers to an interface between the liquid medium and the air. In addition, in a case where water is a main component, the liquid surface refers to a water surface. In addition, when liquid surface-floating culture is performed in the present invention, in some cases, a phenomenon in which a pleat-like structure enters the middle of a liquid from a biofilm on the liquid surface. In the present invention, culturing in such a situation is also included in the liquid surface-floating culture.

Seed algae for performing liquid surface-floating culture may be subjected to suspension treatment, and then, may be added to a culture vessel. Alternately, after adding seed algae to the culture vessel, the seed algae may be stirred in order to accelerate mixing of the seed algae and a liquid medium. In addition, a microalgal biofilm may be added to the liquid surface of a culture vessel to start culturing in a state in which the biofilm is made to float. In addition, the microalgal biofilm may be subjected to division treatment so as not to be sunk as much as possible such that separation of the microalgal biofilm from the liquid surface of the microalgal biofilm minimally occurs, and may be stirred so as to be dispersed on the liquid surface of the culture vessel.

[Pre-Culture Process]

The pre-culture process of the present invention is a process of increasing the number of microalgae until the primary culture can be performed by causing microalgae for storage, which have been obtained after the completion of a purification process, to proliferate. Any well-known culture method can be selected as the culture method of the pre-culture process. For example, a dispersion culture method or an adhesion culture method, liquid surface-floating culture which has been developed by the present inventors, a culture method of the present invention, or the like can be performed. In addition, pre-culture may be performed several times in order for microalgae to proliferate until the microalgae reach a scale in which primary culture can be performed. In addition, in the pre-culture process, stationary culture or non-stationary culture such as shaking culture may be performed in accordance with the purpose.

In addition, culturing can be performed in either of an indoor place or an outdoor place using a culture vessel having a surface area less than or equal to 1 cm² to 1 m².

[Primary Culture Process]

The primary culture process is a culture process which is performed after the pre-culture process is performed and immediately before the final collecting process is performed. The primary culture process can be completed when a sufficient amount of film-like structure or three-dimensional structure is formed on the liquid surface. The primary culture process can be completed in, for example, few days to few weeks, and more specifically, in 5 days to 4 weeks. In addition, the primary culture process may be performed plural times.

In addition, a culture vessel having a surface area greater than or equal to 100 cm² is generally used. It is possible to perform the culturing in either of an indoor place or an outdoor place, but it is preferable to perform the culturing outdoors.

[Seed Algae]

Seed algae of the present invention refer to microalgae which are used when starting the above-described pre-culture process or primary culture process, and become a base for culturing microalgae in the pre-culture process or the primary culture process.

In addition, it is possible to start culturing in a state in which a microalgal biofilm is made to float on the liquid surface or in a state in which microalgae exist on the bottom surface, and even in these cases, it is possible to use the microalgae thereof as seed algae. Furthermore, microalgae which are adhered to and exist on a place other than the bottom surface or the culture vessel, other holding devices constituting the culture vessel can also be used as seed algae.

In addition, it is possible to start the culturing again using microalgae remaining on the liquid surface after the collecting process, as seed algae.

[Use of Microalgae on Liquid Surface as Seed Algae]

In the present invention, culturing can be performed using a microalgal biofilm on the liquid surface as seed algae. There is a method of leaving a part of a microalgal biofilm on the liquid surface in a process of FIG. 1F or 1H. In addition, after a process of FIG. 1G or 1I, culturing can be started by collecting a part of a microalgal biofilm and making the part of the microalgal biofilm float on the liquid surface. Furthermore, culturing can also be started by performing division treatment on the biofilm on the liquid surface in a state of being made to float on the liquid surface as much as possible. In this manner, it is possible to effectively use the liquid surface of a culture vessel and to make microalgae exist even in a region where there are no microalgae. Therefore, in many cases, it is possible to improve the proliferation rate.

In addition, culturing can be started in a state in which a part of the microalgal biofilm is left on the bottom surface and the liquid surface.

[Bottom Surface Algae]

Bottom surface algae of the present invention indicate microalgae existing in the vicinity of the bottom surface of a culture vessel. The bottom surface algae include algae which are adhered to the bottom surface and are not peeled off from the bottom surface by slight liquid flow, or non-adhesive bottom surface algae which exist in the vicinity of the bottom surface and are moved even by light liquid flow. In addition, algae on the liquid surface which have been separated from a microalgal biofilm through a collection operation and have been sunk to the vicinity of the bottom surface can also be included in the non-adhesive bottom surface algae in the present invention.

In the schematic view of the present invention, it is illustrated that the supply of microalgae is performed also from the bottom surface to the top of the liquid surface. However, in reality, there are microalgae in the medium even at locations other than the liquid surface and the bottom surface at a low concentration, and there is a possibility that these microalgae may become a supply source of seed algae. In addition, there are two cases for the supply of microalgae from the bottom surface of the culture vessel to the top of the liquid surface, including a case in which microalgae are moved to the top of the liquid surface without actually being accompanied by proliferation of microalgae on the bottom surface and a case in which the microalgae proliferate while being moved from the bottom surface to the top of the liquid surface.

[Use of Microalgae on Bottom Surface as Seed Algae]

In the present invention, it is possible to continue culturing using microalgae on the bottom surface as seed algae as shown in a process from FIG. 1J to 1C. If there are still nutrient components in a medium, culturing may be continued using a used medium as it is, or a part of the used medium may be discarded and a fresh medium may be added thereto. Regarding the amount of the fresh medium added, the same amount of liquid as the amount of the medium discarded may be added thereto, or a smaller amount or a larger amount of the fresh medium than the amount of the medium discarded may be added thereto. The addition of a fresh medium is more preferable from the viewpoint that it is possible to improve the proliferation rate of microalgae in the primary culture in a later stage.

In a case of using microalgae on the bottom surface as seed algae, a part of bottom surface algae may be peeled off to be dispersed in a medium. This is because, by doing this, it is possible to bring microalgae in a state in which only a part of algal body cannot come into contact with the medium, come into contact with a larger amount of the medium, and therefore, it is possible to favorably improve the proliferation rate.

Non-adhesive microalgae existing on the bottom surface may be removed. This is because if microalgae exist unnecessarily on the bottom surface, decrease in the proliferation rate, which is considered to be caused by unnecessary consumption of nutrient components, can be seen. In addition, the amount of bottom surface algae which exist and are used as seed algae may be adjusted. This is because, by doing this, it is possible to perform appropriate culturing. The quantity of microalgae existing on the bottom surface when starting the culturing is preferably 0.001 μg/cm² to 100 mg/cm², more preferably 0.1 μg/cm² to 10 mg/cm², and most preferably 1 mg/cm² to 5 mg/cm². If the quantity of microalgae thereof is greater than or equal to 0.1 g/cm², it is possible to increase the proportion of the quantity of microalgae before and after the culturing within a short period of time, which is preferable.

[Suspension Treatment]

In the present invention, a sample of microalgae subjected to suspension treatment may be used. This is because, with the suspension treatment, the microalgae in a solution are uniformized and the film thickness after culturing is uniformized, and as a result, in some cases, the quantity of microalgae per culturing area increases. Any well-known method can be used for the suspension treatment, and examples thereof include gentle treatment such as treatment of pipetting or shaking a solution of microalgae put into a container by hand and treatment using a stirrer chip or a stirring rod; strong treatment such as ultrasonic treatment or high speed-shaking treatment; and a method of using a substance such as an enzyme decomposing an adhesion substance such as an intracellular matrix.

[Culture Vessel]

Any well-known shape can be used as the shape of a culture vessel (culture pond) as long as the culture vessel can hold a medium. For example, it is possible to use a culture vessel having a cylindrical shape, a rectangular shape, a spherical shape, a plate shape, a tubular shape, and an irregular shape such as plastic bag. In addition, various well-known methods using types such as an open pond type, a raceway type, and a tube type (J. Biotechnol., 92, 113, 2001) can be used. Examples of the forms that can be used as the culture vessel include culture vessels disclosed in Journal of Biotechnology 70 (1999) 313-321, Eng. Life Sci. 9, 165-177 (2009). Among these, use of the open pond type or the raceway type is preferable in view of costs.

As the culture vessel that can be used in the present invention, any of an open type and a closed type can be used. However, the closed-type culture vessel is preferably used in order to prevent diffusion of carbon dioxide to the outside of the culture vessel when the concentration of carbon dioxide which is higher than that in the air is used. With use of the closed-type culture vessel, it is possible to prevent microorganisms other than microorganisms for culturing, or debris from being mixed in, to suppress evaporation of a medium, and to minimize an influence of wind on a biofilm structure. However, in a case of performing commercial production, culturing in an open system is preferable from the viewpoint that the construction costs are reduced.

[Substrate]

The substrate referred to in the present invention is a solid substance used in FIG. 1F or FIG. 1H. As the shape of the substrate, any shape such as a film shape, a plate shape, a fibrous shape, a porous shape, a convex shape, and a wavy shape may be used. In terms of ease of transfer and ease of collecting microalgae from a substrate, a film shape or a plate shape is preferable.

[Penetrating Structure]

In the method of the present invention, it is possible to use a penetrating structure. With use of this structure, it is possible to significantly decrease the moisture content in a microalgal biomass after collection.

Specifically, there is a method for performing a collection process after a penetrating structure is immersed in a medium and a microalgal biofilm is formed on the liquid surface, the penetrating structure is elevated in a culture vessel and is moved into a gas phase, and is then allowed to stand for a while; or a method for performing collection while continuing the culturing. In addition, collection may also be performed in a state in which, after installing a penetrating structure in a gas phase of a culture vessel, a three-dimensional structure of a microalgal biofilm on the liquid surface is brought into contact with the penetrating structure or is passed through a penetration region of the penetrating structure. Furthermore, there is a method for performing a collection process after allowing a microalgal biofilm to stand for a while by further elevating a penetrating structure in a gas phase in a culture vessel in a state in which the microalgal biofilm is brought into contact with the penetrating structure or is passed through a penetration region; or a method for performing collection while continuing the culturing. Accordingly, it is possible to significantly decrease the moisture content in a collected substance, which is preferable.

The movement of the penetrating structure in a medium and in a gas phase may be performed by adding a medium or by removing a medium. Furthermore, this method and replacement of a medium may be combined.

The penetrating structure used in the present invention has at least one penetration region. The penetration region refers to a region in which a through hole is bored with respect to the structure. Any method may be used in order to form a through hole as the method for forming a through hole. For example, a hole may be bored on a sheet-like substance, or yarn-like materials may overlap each other so as to have a woven fabric shape or a knitted fabric shape.

The number of penetrating films can be installed without any particular restriction. The size thereof may be uniform or non-uniform. As the shape of the penetration region, various forms such as a circular form, a rectangular form, a linear form, and an irregular form can be used. Examples of the penetrating structure include a wire net. In addition, it is preferable that the penetrating structure can be fixed to a culture vessel while not performing the movement.

[Material]

The materials of the culture vessel, the substrate, and the penetrating structure that can be used in the present invention are not particularly limited, and well-known materials can be used. For example, it is possible to use a material formed of an organic polymer compound, an inorganic compound, metal, or a composite thereof. In addition, it is also possible to use a mixture thereof.

Polyethylene derivatives, polyvinyl chloride derivatives, polyester derivatives, polyamide derivatives, polystyrene derivatives, polypropylene derivatives, polyacrylic derivatives, polyethylene terephthalate derivatives, polybutylene terephthalate derivatives, nylon derivatives, polyethylene naphthalate derivatives, polycarbonate derivatives, polyvinylidene chloride derivatives, polyacrylonitrile derivatives, polyvinyl alcohol derivatives, polyethersulfone derivatives, polyarylate derivatives, allyl diglycol carbonate derivatives, ethylene-vinyl acetate copolymer derivatives, fluorine resin derivatives, polylactic acid derivatives, acrylic resin derivatives, ethylene-vinyl alcohol copolymers, ethylene-methacrylic acid copolymers, and the like can be used as the organic polymer compound.

Glass, ceramics, concrete, and the like can be used as the inorganic compound.

Alloys such as iron, aluminum, copper, or stainless steel can be used as a metallic compound.

Among the above, it is preferable that a part of the material of the substrate or the culture vessel is formed of at least one selected from glass, polyethylene, polypropylene, nylon, polystyrene, vinyl chloride, and polyester.

In addition, the materials of the culture vessel, the substrate, and the penetrating structure may be the same as each other or different from each other.

In addition, in a case of using a closed-type culture vessel, the light receiving surface may be made of a material through which light is transmitted, and a transparent material is more preferable.

[Medium (Liquid Medium)]

In the present invention, any well-known medium (liquid medium) can be used as long as it is possible to culture microalgae. Examples of well-known media include an AF-6 medium, an Allen medium, a BBM medium, a C medium, a CA medium, a CAM medium, a CB medium, a CC medium, a CHU medium, a CSi medium, a CT medium, a CYT medium, a D medium, an ESM medium, an f/2 medium, an HUT medium, an M-11 medium, an MA medium, an MAF-6 medium, an MF medium, an MDM medium, an MG medium, an MGM medium, an MKM medium, an MNK medium, an MW medium, a P35 medium, a URO medium, a VT medium, a VTAC medium, a VTYT medium, a W medium, a WESM medium, an SW medium, and an SOT medium. Among these, freshwater media are an AF-6 medium, an Allen medium, a BBM medium, a C medium, a CA medium, a CAM medium, a CB medium, a CC medium, a CHU medium, a CSi medium, a CT medium, a CYT medium, a D medium, an HUT medium, an M-11 medium, an MA medium, an MAF-6 medium, an MDM medium, an MG medium, an MGM medium, an MW medium, a P35 medium, a URO medium, a VT medium, a VTAC medium, a VTYT medium, a W medium, an SW medium, and an SOT medium. As media for culturing the above-described AVFF007 strains, a C medium, a CSi medium, and a CHU medium, and a mixture of these media are preferable. It is desirable to select the medium in accordance with the types of microalgae to be cultured.

The media may be subjected to ultraviolet ray sterilization, autoclave sterilization, and filter sterilization, or may not be sterilized.

Different media may be used as media in the pre-culture process and the primary culture process. In addition, a medium may be changed to another medium during the culture processes.

[Carbon Dioxide]

It is necessary to supply carbon dioxide for culturing a large quantity of microalgae.

In a case of performing dispersion culture in a pre-culture process, carbon dioxide may be supplied to a medium through bubbling which is a conventional method. However, in a case of performing liquid surface-floating culture, it is preferable to supply carbon dioxide from a gas phase. This is because, if carbon dioxide is supplied to a medium through a method such as bubbling, there is a possibility that the structure of a microalgal biofilm on the liquid surface may be destroyed, unevenness in the quantity of algal bodies may occur, the efficiency of collecting a biofilm on a substrate through a collecting process may be deteriorated, and therefore, the quantity of algal bodies collected may decrease.

In the present invention, it is possible to use carbon dioxide in the air, but it is also possible to use carbon dioxide having a higher concentration than that in the air. In this case, it is desirable to perform culturing in a closed-type culture vessel or in a culture vessel which is covered with a coating material such as an agricultural film, in order to prevent the loss of carbon dioxide due to diffusion. The concentration of carbon dioxide in this case is not particularly limited as long as it is possible to achieve the effect of the present invention, but is preferably greater than or equal to the concentration of carbon dioxide in the air and less than 20 volume %, more preferably 0.01 volume % to 15 volume %, and still more preferably 0.1 volume % to 10 volume %. In addition, carbon dioxide may be discharged using a combustion device. In addition, carbon dioxide may also be generated using a reagent.

[Light Source and Amount of Light]

As light sources that can be used in the present invention, any known light source can be used. However, it is possible to use sunlight, LED light, a fluorescent lamp, an incandescent lamp, xenon lamp light, a halogen lamp, and the like. Among these, it is preferable to use sunlight as natural energy, an LED having a good luminous efficiency, or a fluorescent lamp that can be simply used.

The amount of light is preferably 100 lux to 1000000 lux and more preferably 300 lux to 500000 lux. The most preferable amount of light is 1000 lux to 200000 lux. If the amount of light is greater than or equal to 1000 lux, it is possible to culture microalgae, and if the amount of light is less than or equal to 200000 lux, there is a little adverse effect on culturing due to photolesion.

Light may be radiated through any method such as continuous irradiation, and repetition of irradiation and non-irradiation at a constant time interval, but it is preferable that light be turned on and off at a time interval of 12 hours.

The wavelength of light is not limited, and any wavelength can be used as long as the wavelength is a wavelength at which photosynthesis can be performed. A preferred wavelength is a wavelength of sunlight or a wavelength similar to that of sunlight. An example in which the growth rate of photosynthetic organisms is improved by radiating a single wavelength has been reported, and even in the present invention, it is also possible to use such an irradiation method.

[Other Culture Conditions]

In the present invention, the pH of a liquid medium (hereinafter, the liquid medium is also referred to as a medium) used in a pre-culture process or a primary culture process is preferably within a range of 1 to 13, more preferably within a range of 3 to 11, still more preferably within a range of 5 to 9, and most preferably within a range of 6 to 8.

In addition, it is preferable to select the pH of the medium in accordance with the types of microalgae since a preferred pH is changed in accordance with the types of microalgae. The pH of the liquid medium refers to the pH when starting the culturing. In addition, in some cases, the pH during a culture process is changed accompanying the culturing, and therefore, the pH during the culture process may be changed.

In the present invention, it is possible to add a substance, which has a buffer action, to a medium for maintaining a constant pH in the medium. Accordingly, in some cases, it is possible to suppress a problem in which the pH in a medium is changed in accordance with the progress of culturing of microalgae, or to suppress the phenomenon in which the pH is changed due to supply of carbon dioxide to the medium. As the substance having a buffer action, it is possible to use a well known substance. The use thereof is not limited, but it is possible to suitably use 4-(2-hydroxyethyl)-1-piperazineethanesulfonic acid (HEPES), a sodium phosphate buffer solution, a potassium phosphate buffer solution, or the like. The concentrations or the kinds of buffer substances can be determined in accordance with the types or the culture environments of the microalgae.

If the water depth of a liquid medium in a case where dispersion culture is performed, light does not reach the depth and there is a problem that the stirring efficiency is deteriorated, and therefore, there is restriction. However, in a case of liquid surface-floating culture, microalgae proliferate on the liquid surface at high density, and therefore, it is unnecessary to supply light to the depth of a culture vessel. Moreover, since stirring is not basically performed, it is possible to make the water depth shallow. Accordingly, in terms of the amount of water used becoming small and the handling efficiency becoming good, it is preferable to make the water depth shallow. The water depth is preferably greater than or equal to 0.4 cm, more preferably 1 cm to 10 m, more preferably 2 cm to 1 m, and most preferably 4 cm to 30 cm. If the water depth is greater than or equal to 0.4 cm, it is possible to form a biofilm, and if the water depth is less than or equal to 10 m, the handling becomes easy. If the water depth is 4 cm to 30 cm, an influence due to evaporation of moisture is minimized and the handling of a solution containing a medium or microalgae becomes easy.

The culture temperature can be selected in accordance with the types of microalgae and is not particularly limited. However, the culture temperature is preferably 0° C. to 90° C., more preferably 15° C. to 50° C., and most preferably greater than or equal to 20° C. and less than 40° C. If the culture temperature is greater than or equal to 20° C. and less than 40° C., it is possible to make microalgae suitably proliferate.

If the lower limit input microalgae quantity of microalgae, that is, the quantity of microalgae used when starting culturing is one within a range of culturing, microalgae can proliferate as long as time is given, and therefore, there is no particular restriction. However, the lower limit input microalgae quantity thereof is preferably greater than or equal to 1 cell/cm³, more preferably greater than or equal to 1000 cells/cm³, and still more preferably greater than or equal to 1×10⁴ cells/cm³. Regarding the upper limit input microalgae quantity of microalgae, microalgae can basically proliferate at any high concentration, and therefore, there is no particular restriction. However, the upper limit input microalgae quantity thereof is preferably lower than or equal to 1×10⁹ cells/cm³, more preferably lower than or equal to 1×10⁸ cells/cm³, and still more preferably lower than or equal to 5×10⁷ cells/cm³ from the viewpoint that, when the concentration thereof is higher than or equal to a certain concentration, the ratio of the number of microalgae after the proliferation to the number of input microalgae decreases as the quantity of microalgae becomes higher.

The pre-culture period and the primary culture period in the present invention can be selected in accordance with the types of microalgae and is not particularly limited. However, the pre-culture period and the primary culture period is preferably 1 day to 300 days, more preferably 3 days to 100 days, and still more preferably 7 days to 50 days.

[Repetitive Culturing of Microalgal Biofilm]

In the present invention, after collecting a microalgal biofilm, culturing can be performed again using microalgae, which remain on the bottom surface or in other regions, as seed algae many times as long as nutrient components for proliferation remain in the medium.

However, there is a high possibility that the proliferation rate becomes slow if the concentration of microalgae is too low. Therefore, in such a case, it is possible to newly add a medium thereto, to replace at least a part of a medium with another medium, and to add solid nutrient components or highly concentrated nutrient components to a medium.

[Multistage Culture]

Culturing in the present invention can be performed as multistage culture in which culturing is performed by overlapping at least two culture vessel each other. In the multistage culture, the culturing stage in one culture vessel includes an induction phase, a logarithmic proliferation phase, a useful substance accumulation phase, or a culture stop phase, and may be performed in a manner different from a culturing stage in the other culture vessel. In addition, culturing using a culture vessel in an upper stage may be performed in order to provide seed algae and culturing using a culture vessel in a lower stage may be performed in order to provide a useful substance.

Furthermore, in the multistage culture, culturing may be performed in the upper stage using light and culturing may be performed in the lower stage mainly using sugar without using light unlike in the upper stage. Accordingly, it is possible to improve a problem in that the amount of light is deteriorated in the lower stage, and as a result, the amount of proliferation of microalgae is deteriorated.

In the multistage culture, a light source and light guiding means may be used in order to supply light.

[Size and Proliferation Rate of Microalgal Biofilm Formed on Liquid Surface]

The size of a microalgal biofilm is preferably greater than or equal to 0.1 cm², more preferably greater than or equal to 1 cm², still more preferably greater than or equal to 10 cm², and most preferably the same as the area of the liquid surface of a culture vessel. If the size thereof is greater than or equal to 0.1 cm², it is possible to increase the ratio of the quantity of microalgae when finishing culturing to the quantity of microalgae when starting culturing within a short period of time, which is preferable.

In addition, a plurality of microalgal biofilms may exist within a culture region.

The thickness of a microalgal biofilm is preferably within a range of 1 μm to 10000 m, more preferably within a range of 1 m to 1000 m, and most preferably within a range of 10 m to 1000 m. If the thickness thereof is within a range of 10 m to 1000 m, it is possible to harvest a sufficient amount of biofilm with high strength.

In a case where the biofilm according to the present invention is a steric three-dimensional structure which is formed such that a part or a plurality of parts of a film-like structure rise in a bubble shape, in general, the height of the three-dimensional structure having a liquid surface of a medium as a reference is preferably within a range of 0.01 mm to 100 mm, more preferably within a range of 0.1 mm to 20 mm, and most preferably within a range of 5 mm to 20 mm. If the height thereof is within a range of 5 mm to 20 mm, it is possible to sufficiently decrease the moisture content and to suppress the height of a culture vessel to be low.

In addition, microalgae according to the present invention preferably have a high proliferation rate on the liquid surface. The proliferation rate (that is, an average proliferation rate per day during a period of a logarithmic proliferation phase) of microalgae in the logarithmic proliferation phase is preferably greater than or equal to 0.1 g/m²/day by dry weight, more preferably greater than or equal to 0.5 g/m²/day by dry weight, still more preferably greater than or equal to 1 g/m²/day by dry weight, and most preferably greater than or equal to 3 g/m²/day by dry weight. The proliferation rate of microalgae in the logarithmic proliferation phase is generally less than or equal to 1000 g/m²/day by dry weight.

The weight of dry alga bodies of the biofilm according to the present invention per unit area is preferably greater than or equal to 0.001 mg/cm², more preferably greater than or equal to 0.1 mg/cm², and particularly preferably greater than or equal to 1 mg/cm². The most preferred weight of the dry alga bodies is greater than or equal to 5 mg/cm². This is because it is expected that if the weight of the dry alga bodies per unit area is great, the amount of biomass, such as oil, which has been obtained will become great. In general, the weight of the dry alga bodies of a biofilm per unit area is less than or equal to 100 mg/cm².

Microalgae capable of forming a biofilm, which has the above-described structure, or the area, the thickness, the height, the proliferation rate, and the dry weight of the algal bodies per unit area within the above-described ranges, on the liquid surface, are preferable as the microalgae of the present invention for the same reasons described above.

[Collection]

It is possible to collect a microalgal biofilm on the liquid surface in a state in which the liquid surface in the culture vessel is partially covered with the biofilm, but it is preferable to collect the biofilm after the entirety of the liquid surface in the culture vessel is covered with the biofilm in view of obtaining a large quantity of algal bodies of microalgae. In addition, the collection may be performed after continuing culture for a while after the entirety of the liquid surface is covered with the biofilm.

Particularly, it is preferable to perform the collection after a three-dimensional structure is formed on the liquid surface. The three-dimensional structure is a structure which can be seen when a film-like structure further proliferates. In the three-dimensional structure, a larger quantity of microalgae which can be collected and lower moisture content are preferable in comparison to the two-dimensional film-like structure.

As the above-described collection method, greater than or equal to 70% of a biofilm formed on the liquid surface is preferably collected, greater than or equal to 80% thereof is more preferably collected, greater than or equal to 90% thereof is still more preferably collected, and 100% thereof is most preferably collected. The collection rate of a biofilm formed on the liquid surface can be confirmed by, for example, being visually recognized.

In addition, only a biofilm on the liquid surface may be collected, or both of at least a part of the biofilm on the liquid surface or at least a part of microalgae on the bottom surface may be collected. This is because it is possible to use both of the microalgae on the liquid surface and the microalgae on the bottom surface as biomass. However, in general, when considering oil as a useful substance, the content of oil of the microalgae on the liquid surface becomes higher than that of the microalgae on the bottom surface. Accordingly, the collection of bottom surface algae may be avoided as much as possible.

[Collection of Microalgal Biofilm on Liquid Surface Using Transferring Method]

The transferring method is a process of transferring a microalgal biofilm (film-like structure or three-dimensional structure) on the liquid surface to a first substrate as shown in FIGS. 1F and 1G, and is a type of adhesion which is performed without substantial proliferation. The first substrate is gently inserted with respect to the liquid surface so as to be parallel to or at an angle close to the liquid surface, and the microalgal biofilm on the liquid surface is adhered to surface of the first substrate. When performing the insertion, the first substrate is slightly obliquely inserted with respect to the liquid surface, and is then finally made to be parallel to the liquid surface. Then, it is possible to collect a large amount of biofilm with a smaller number of times of transferring, which is preferable. The transferring may be performed plural times in terms of improving the transferring efficiency.

[Collection of Microalgal Biofilm on Liquid Surface Using Deposition Method]

The method for collecting microalgae on the liquid surface using a second substrate as shown in FIG. 1H is a collection method using a deposition method. The method is a method for vertically or obliquely inserting the second substrate with respect to a microalgal biofilm on the liquid surface of a culture vessel, inserting and pulling the second substrate so as to follow the surface of the biofilm, and collecting the biofilm while depositing the biofilm on the surface of the second substrate, as shown in the drawing.

In the drawing, the second substrate is moved from the right side to the left side, but the movement direction of the second substrate may be a reverse direction (that is, movement from the left side to the right side), and the collection may be performed plural times. This is because the collection rate is improved by performing the collection plural times. In a case of performing the collection plural times, the second substrate may be used in a state in which a biofilm is adhered to the second substrate as it is, or a new second substrate may be prepared and used. In addition, although only one sheet of a second substrate is illustrated in FIGS. 1A to 1J, a plurality of sheets of the second substrates may be simultaneously used. Accordingly, the collection rate is improved. Among these, it is preferable to remove a collected biofilm using one sheet of the second substrate, and then, to use the one sheet of the second substrate for the next collection as much as the strength of the second substrate allows, from the viewpoint of the installation costs of a collection device. In addition, the size of the second substrate, the angle or the movement rate of the second substrate with respect to the liquid surface, or the like can be freely set in accordance with the purpose. FIG. 1G is in a state in which a biofilm is collected on the second substrate.

The size of the second substrate can be appropriately changed in accordance with the size of a culture vessel. However, it is preferable to use a second substrate having a size slightly smaller than that of the minor diameter of the inner wall of the culture vessel. This is because, by doing this, it is possible to avoid unnecessary contact of the second substrate with the inner wall of the culture vessel when moving the second substrate, and an escape of a microalgal biofilm on the liquid surface during collection via a gap between the culture vessel and the second substrate is hardly generated.

In addition, in some cases, a microalgal biofilm proliferating on the liquid surface within a culture vessel grows from a film shape to a pleat shape depending on the culturing state. In this case, it is possible to collect the pleat-like biofilm by making the insertion depth of the second substrate into a liquid deep.

[Detachment of Microalgal Biofilm from Substrate]

Detachment is a part of a collection process.

Any method may be used as the method for detaching a microalgal biofilm from a substrate as long as the method used is a method in which it is possible to peel off microalgae on a substrate. It is possible to peel off a microalgal biofilm from a substance by applying a stream of water; subjecting a container, into which a substrate is put, to ultrasonic treatment; vigorously shaking the container, into which a substrate is put, after closing a lid of the container; performing high speed-shaking treatment; or using a cell scraper. Among these, a method for peeling off a microalgal biofilm from a substrate using a holding device, for which a material which does not damage a substrate is used, for example, a cell scraper, is preferable. Furthermore, it is also possible to peel off a microalgal biofilm from the top of a substrate by simply inclining the substrate. This method is the most preferred method due to its simplicity. In addition, the substrate may be used again many times.

In addition, in the schematic view of FIGS. 1A to 1J, the microalgal biofilm is detached from the substrate after taking the substrate out of the culture vessel, but the microalgal biofilm may be detached from the substrate within the culture vessel.

[Dry Alga Bodies]

The dry alga bodies in the present invention are obtained by drying a collected substance of microalgae which has been obtained by the present invention.

Any well-known method can be used as the method for drying the collected substance of microalgae as long as it is possible to reduce the moisture in the collected substance of microalgae, and there is no particular restriction. Examples thereof include a method of sun-drying a collected substance of microalgae; a method of heat-drying a collected substance of microalgae; a method of freeze-drying a collected substance of microalgae; and a method of blowing dry air onto a collected substance of microalgae. Among these, the freeze-drying method is preferable in view of being capable of suppressing decomposition of components contained in a collected substance of microalgae, and the heat-drying method or the sun-drying method is preferable in view of being capable of efficiently perform the drying in a short period of time.

[Moisture Content]

The moisture content of the present invention is obtained by dividing the weight of moisture contained in a collected substance by the weight of the collected substance, and then by multiplying 100. The moisture content of a microalgal biofilm in the present invention is preferably 99% to 60%, more preferably 95% to 80%, and most preferably 90% to 85%. In a case where culturing is performed using a penetrating structure, there is no restriction.

The moisture content in a case where microalgae are collected using a centrifugal separator after being cultured through dispersion culture is generally set to about 90%, and the moisture content of a biofilm on the liquid surface obtained through a culture method in the present invention is lower than the value, which is excellent compared to a conventional method. The moisture content of a three-dimensional structure is lower than that of a film-like structure. It is estimated that this is because the three-dimensional structure is away from the liquid surface and is close to a light source, and therefore, drying is progressed to some extent.

[Useful Substance]

The useful substance in the present invention is one type of biomass derived from microalgae and is the name of a substance beneficial to an industry which is obtained through a process such as an extraction process from biomass and a purification process. Such a substance includes a final product, an intermediate, or a raw material of a pharmaceutical product, cosmetics, or a health food, or the like; a raw material, an intermediate, or a final product of a chemical synthetic substance; a hydrocarbon compound; an energy-alternative substance such as oil, alcohol compound, hydrogen, or methane; an enzyme; protein; nucleic acid; a lipid compound such as sugar or DHA; and astaxanthin. The useful substance can be accumulated in microalgae through a useful substance accumulation process.

[Biomass and Oil]

The biomass in the present invention refers to a renewable organic resource derived from organisms excluding a fossil resource, and examples thereof include substances, food products, materials, fuel, and resources derived from organisms. As the algal biomass, residues of microalgae after the microalgae itself (which may also have a biofilm shape) and the useful substance have been collected are included.

Oil in the present invention refers to a flammable fluid substance, is a compound mainly formed of carbon and hydrogen, and is a substance occasionally containing oxygen atom, nitrogen atom, and the like. In general, oil is a mixed substance and is a substance which is extracted using a low-polarity solvent such as hexane or acetone. There is a case in which the composition thereof is formed of hydrocarbon compounds, fatty acids, triglycerides, or the like, and a case in which oil is formed of a plurality of types of compositions selected therefrom. In addition, oil can be esterified to be used as biodiesel.

The method of collecting a useful substance and oil contained in a collected substance of microalgae is not particularly limited as long as the method does not impair the effect of the present invention.

As a general method for extracting oil, dry alga bodies are obtained by heat-drying a final collected substance, and then, oil is extracted using an organic solvent after performing cell-disruption. In general, the extracted oil contains impurities such as chlorophyll, and therefore, purification is performed. There is a case of performing purification through silica gel column chromatography or performing purification through distillation (for example, a distillation method disclosed in JP2010-539300A). In the present invention, it is possible to use such a method as well.

In addition, there is also a method of extracting oil in algal bodies using an organic solvent after crushing microalgae through an ultrasonic treatment or dissolving microalgae using protease or an enzyme (for example, a method disclosed in JP2010-530741A). In the present invention, it is possible to use such a method as well.

In addition, it is preferable that the biofilm according to the present invention has high oil content in view of usefulness as biomass. Specifically, the oil content per dry alga body of the biofilm is preferably higher than or equal to 5 mass %, more preferably higher than or equal to 10 mass %, and particularly preferably higher than or equal to 15 mass %. In general, the oil content per dry alga body of the biofilm is lower than or equal to 80 mass %.

### EXAMPLE

The present invention will be further described in detail with reference to the following Examples, but the present invention is not limited to the following Examples.

### Example 1

### Nitrogen Compound-Cut Culture

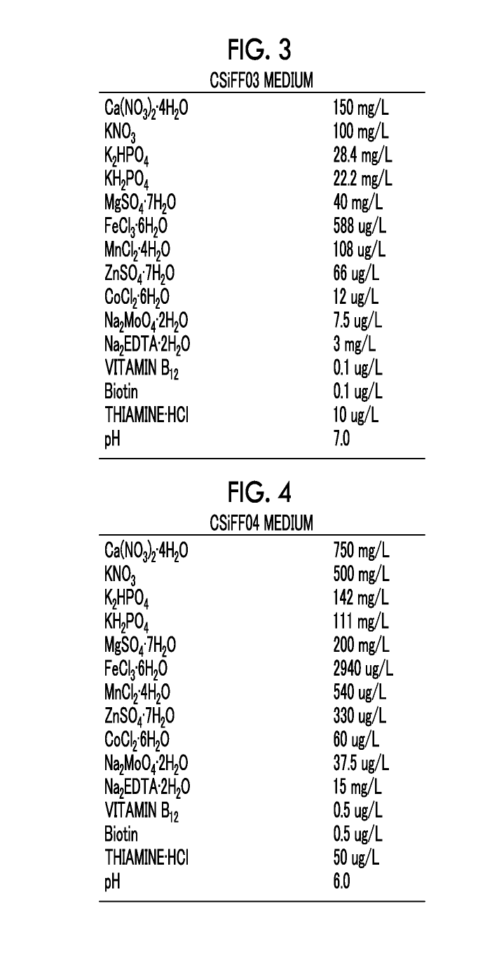

The input alga body concentration of AVFF007 strains of microalgae is adjusted to 1×10⁵ cells/mL, and a pre-culture process is performed. Liquid surface-floating culture was performed under the conditions for stationary culture by preparing a suspension liquid of the above-described microalgae using a CSiFF03 medium having the composition shown in FIG. 3, putting 55 mL of the prepared suspension liquid into a Purobio Petri dish (2-4727-01, As One Corporation), and installing this in a plant bioshelf for tissue culture (AV152261-12-2, Ikeda Scientific Co., Ltd). Culturing was performed at room temperature (23° C.) by performing light irradiation by turning on and off a fluorescent lamp at 4000 lux every 12 hours. Collection of a microalgal biofilm formed on the liquid surface was performed using a polyethylene film.

The collected biofilm was set in a beads cell disrupter MS-100 (Tomy Seiko Co., Ltd.) after putting a small amount of CSiFF04 medium (FIG. 4) into a 5 mL tube for homogenizing (TM-655, Tomy Seiko Co., Ltd); and homogenization treatment lasting for 20 seconds was performed three times at 4200 rpm to obtain a suspension liquid a of microalgae. However, beads were not used.

The suspension liquid a was diluted and the turbidity was calculated by measuring the absorbance at 660 nm. 970 mL of a suspension liquid b at a concentration of 5×10⁵ cells/mL was obtained by calculating the quantity of algal bodies from a relational expression between the turbidity and the number of algal bodies which have been calculated in advance and diluting the suspension liquid a with the CSiFF04 medium.

Liquid surface-floating culture was performed as first primary culture after 40 mL of the suspension liquid b was put into a polystyrene case no. 28 (4-5605-05, As One Corporation) which was put into a vacuum desiccator (1-070-01, As One Corporation), and the concentration of carbon dioxide was set to 5%. As the other culture condition, stationary culture was performed at room temperature (23° C.) by performing light irradiation by turning on and off a fluorescent lamp which was set to 15000 lux every 12 hours. Light shielding plates were installed on the bottom surface and a side surface and 16 polystyrene cases no. 28 were used in total.

The following treatment was performed 14 days after the start of the culturing.

### Example 1-a

A biofilm of water surface algae on a polystyrene case no. 28 after the culturing was collected through deposition method using a nylon film as a second substrate. The weight of the collected substance was measured, the weight of the collected substance after freeze-drying was further measured, and the mass of the collected substance corresponding to medium components was reduced, and then, the dry weight and the moisture content were calculated. An average value of each of the quantity of algal bodies of four samples was calculated, and as a result, the average value thereof was 4.66 mg/cm².

### Example 1-b

A medium in a region, in which there are substantially no microalgae between water surface algae on a polystyrene case no. 28 after culturing, and bottom surface algae, was suctioned as much as possible using a 1 mL long tip. The long tip was inserted into the medium by destroying a part of the water surface algae. The microalgae on the surface of water were brought into contact with microalgae on the bottom surface with almost no destruction. Subsequently, 35 mL of a fresh CSiFF04 (N-) (FIG. 6) medium was added to the polystyrene case no. 28 using 1 mL long tip so as not to disturb the structure of the water surface algae as possible. In this process, the water surface algae which were brought into contact with the bottom surface are separated from the bottom surface in accordance with the addition of the medium, and the level of which was elevated while the water surface algae floated on the surface of water in accordance with the elevation of the surface of water. The same film-like structure as that before the replacement of the medium was almost formed when visually observed. Accordingly, in a series of processes, there was almost no disturbance of the basic structure except for the region into which the 1 mL long tip was inserted. In this process, collection of the water surface algae was not performed. In addition, the number of samples was 4.

### Example 1-c

Medium replacement was performed through the same process as that in Example 1-b. However, a CSiFF04 medium was used here.

### Example 1-d

Medium replacement was not performed.

After performing the treatments of Examples 1-b, 1-c, and 1-d, culturing was performed as second primary culture under the same conditions as those in first primary culture.

7 days after the culturing, collecting of microalgal biofilms on the surfaces of water with respect to all of the samples was performed through the same method as that in Example 1-a.

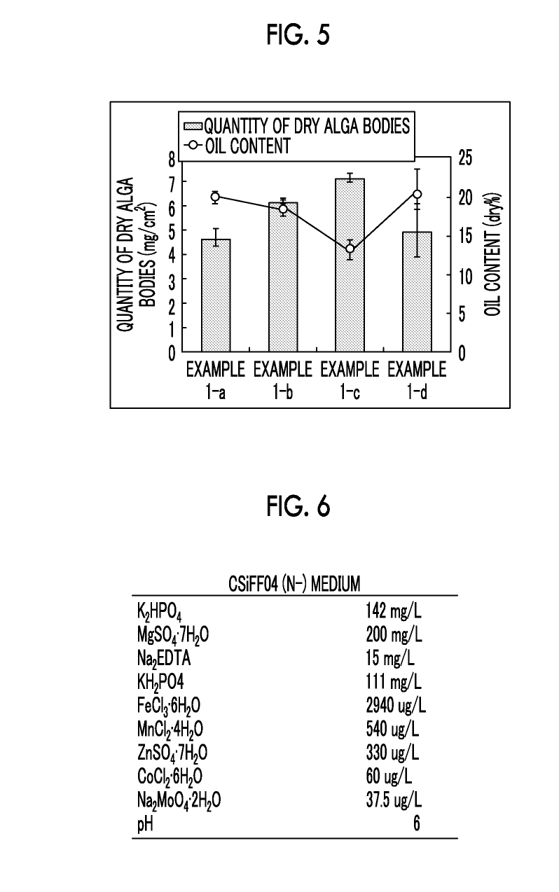

The results were shown in FIG. 5. The quantity of algal bodies was highest in Example 1-c to which nutrient components were supplied by supplying the fresh medium, and subsequently high in Example 1-b in which the medium was replaced with the CSiFF04 (N-) medium. The quantity of algal bodies in Examples 1-a was almost the same as that in Example 1-d.

After disrupting collected microalgae using a cell disrupter, oil was extracted through a hexane extraction method, and the oil content (g/g dry weight, dry %) was obtained. The oil content was smallest in Example 1-c, and the oil contents in other Examples were the same as each other.

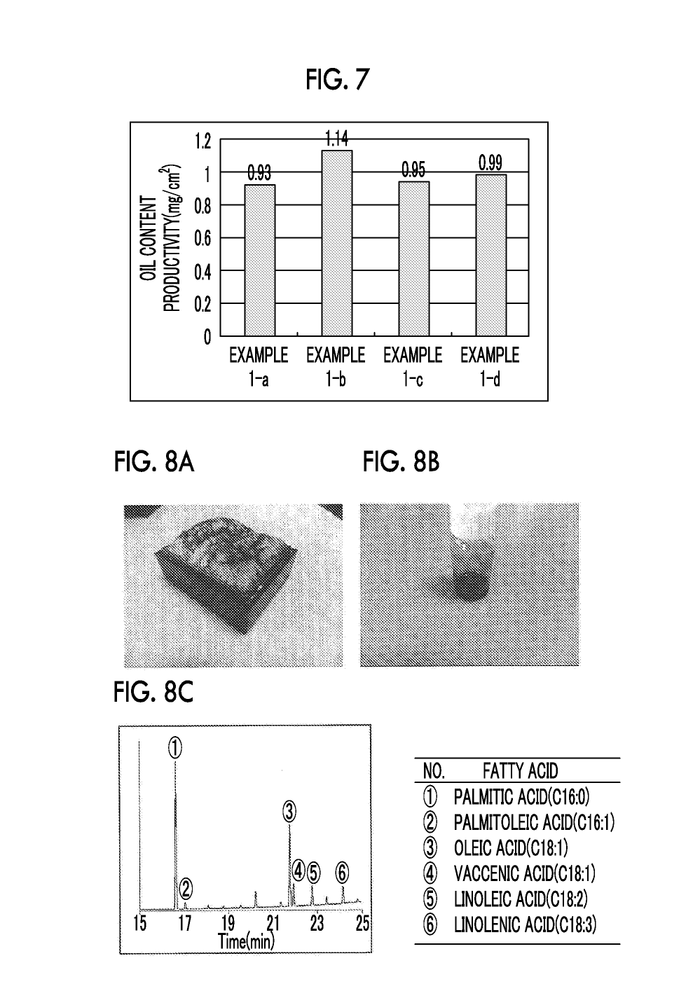

In FIG. 7, results of oil productivity calculated by multiplying the dry weight and the oil content are shown. A result showing the most favorable value was a result of Example 1-b in which the medium was replaced with the CSiFF04 (N-) medium. Other Examples had almost the same oil productivity as each other. From the above, it was found that it was possible to improve the oil productivity by replacing a medium with a medium from which a nitrogen compound was removed.

Furthermore, culturing was performed through the same method as that in Example 1-b using FFG039 strains as microalgae. However, 16 polystyrene cases no. 28 were used. The state after the culturing is shown in FIG. 8A. After collecting this sample through a deposition method, freeze-drying was performed, and the moisture content which was calculated became 86%. Furthermore, the oil content became 35 dry % (FIG. 8B). Furthermore, an analysis was performed using a GC-MS spectrum, and as a result, palmitic acid and oleic acid were main products. Analysis of hydrocarbon was also performed, but the amount analyzed was very small.

### Example 2

### Improvement of Yield of Oil Through Addition of Distilled Water

Similarly to Example 1, pre-culture and first primary culture were performed. However, the first primary culture was performed in 70 mL of a suspension liquid b of algal bodies, that is, in a water depth of 1 cm using a staining tray (1-1413-01, As One Corporation) instead of a polystyrene case no. 28 as a culture vessel. In addition, 6 culture vessels were prepared.

14 days after the culturing, 70 mL (water level of 2 cm after addition) of distilled water was added to each two culture vessels out of the six culture vessels. Furthermore, 280 mL (water level of 5 cm after addition) of distilled water was added to each two culture vessels. The NO₃ concentration before the addition of distilled water was 701 mg/L, 350 mg/L in a case where 70 mL of distilled water was added, 140 mg/L in a case where 280 mL of distilled water was added.

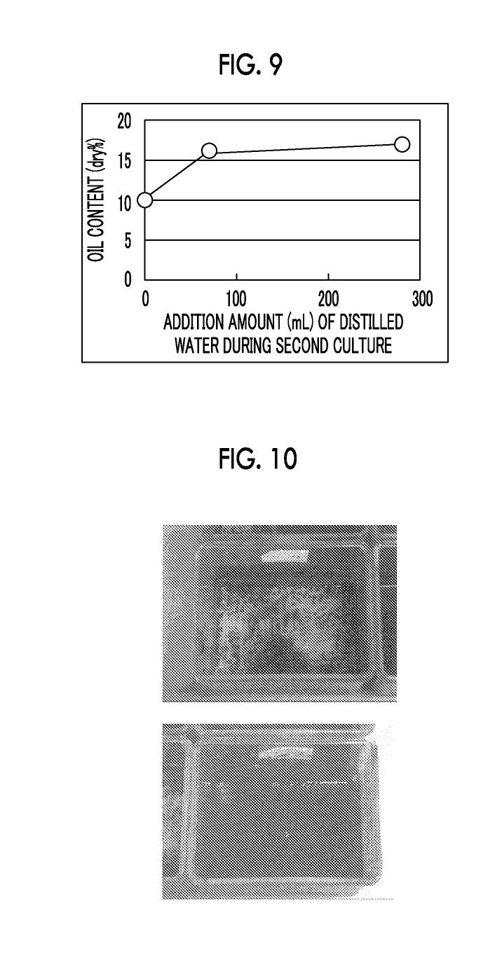

Second primary culture was performed through the same method as that in Example 1, microalgae on the surface of water were collected through the same method as that in Example 1, and freeze-drying and measurement of the weight were performed. The results were shown in FIG. 9. The oil content was more increased as the amount of distilled water added during the second primary culture was larger. It is considered that the amount of oil was increased as the same reason as the effect of replacing a medium with a medium which does not including no nitrogen compound of Example 1.

### Example 3

### Improvement of Recovery Properties Through Addition of Distilled Water

Pre-culture and first primary culture were performed through the same method as that in Example 2. However, when performing the culturing, the amount of medium was 105 mL, that is, the water depth was 1.5 cm, and a total of 4 culture vessels were used.

14 days after the culturing, 245 mL of distilled water was added to each two culture vessels out of four culture vessels. That is, the water depth became 5 cm. The collected amount became 7.4 mg/cm² in all cases. However, in a case where not distilled water was added to culture vessels as shown in FIG. 10, a part of bottom surface algae was collected, and there was no such collected amount.

### Example 4

### Method of Peeling Off Contact Region Between Algae on Surface of Water and Wall Surface

Pre-culture and first primary culture were performed through the same method as that in Example 2. However, when performing the culturing, the amount of medium was 350 mL, that is, the water depth was 5 cm, and a total of 8 culture vessels were used. The culturing was performed using FFG039 strains, as the species of algae.

14 days after the culturing, regarding each set of four culture vessels out of the eight culture vessels, a microalgal biofilm adhering on the wall surface of a region, in which the liquid surface and the wall surface were brought into contact with each other, was peeled off using a metal spatula. At this time, the biofilm was peeled off from the wall surface while being made to float on the liquid surface without destroying the structure of the biofilm as possible.

Next, a medium was removed as much as possible from all of the culture vessels and 40 mL of a CSiFF04 (N-) medium was put into all of the culture vessels. At this time, in samples in which microalgae adhering on the wall surface were not peeled off, microalgal biofilm on the liquid surface was destroyed in accordance with the removal of the medium. A part of the biofilm adhered on the wall surface or the biofilm was destroyed. In contrast, regarding the samples in which microalgae adhering on the wall surface were peeled off, the biofilm on the liquid surface was sunk together with the liquid surface while floating on the liquid surface as it is in its original condition without being caught halfway.

Furthermore, the dry weight of microalgae on the surface of water after the culturing was performed for 7 days was obtained. The dry weight of the samples in which the adhesion of microalgae at a contact point with the wall surface was not peeled off became 5.7 mg/cm² and the quantity of dry alga bodies of the samples in which the adhesion of microalgae at a contact point with the wall surface was peeled off became 6.2 mg/cm². The quantity of algal bodies in the samples in which the adhesion of microalgae at a contact point with the wall surface was large. It is estimated that this is because the adhesion of microalgae in the former case was unnecessary, and as a result, the quantity of microalgae on the surface of the water was decreased.

### Example 5

### Sugar-Containing Medium

As pre-culture, stationary culture was performed at a concentration of carbon dioxide of 5% at 23° C. under irradiation with a fluorescent lamp (light irradiation by turning on and off the fluorescent lamp every 12 hours) at 15000 lux after a mixture of 40 mL of a CSiFF04 medium (FIG. 4) and AVFF007 strains (at a concentration of algal bodies of 5×10⁵ cells/mL) was put into a polystyrene case no. 28, which was then put into a vacuum desiccator. The side surface and the bottom surface of the polystyrene case no. 28 were covered with plastic cases.

14 days after the culturing, a culture vessel was taken out of the vacuum desiccator, and a microalgal biofilm on the surface of water of the medium was collected through a deposition method using a nylon film having the same length as a short side of the polystyrene case no. 28. The biofilm was put into a 5 mL tube for homogenizing together with a small amount of CSiFF04 medium, the tube for homogenizing was set in a beads-type cell disrupter MS-100, and homogenization treatment lasting for 20 seconds was performed three times at 4200 rpm to obtain a suspension liquid a of microalgae. However, beads were not used.

This solution was diluted and the turbidity was calculated by measuring the absorbance at 660 nm. 170 mL of a suspension liquid b at a concentration of 5×10⁵ cells/mL was obtained by calculating the quantity of algal bodies of the above-described suspension liquid a from a relational expression between the turbidity and the number of algal bodies which have been calculated in advance, and diluting the suspension liquid a with the medium. As the medium, a CSiFF04 medium containing 0 mg/mL to 10 mg/mL of glucose was used.

Culturing was performed under the same culture conditions as those in the pre-culture, and a microalgal biofilm on the surface of water was collected through a deposition method using a nylon film on days 8 and 14 after the start of the culturing. Freeze-drying of the collected substance was performed and the dry weight was calculated.

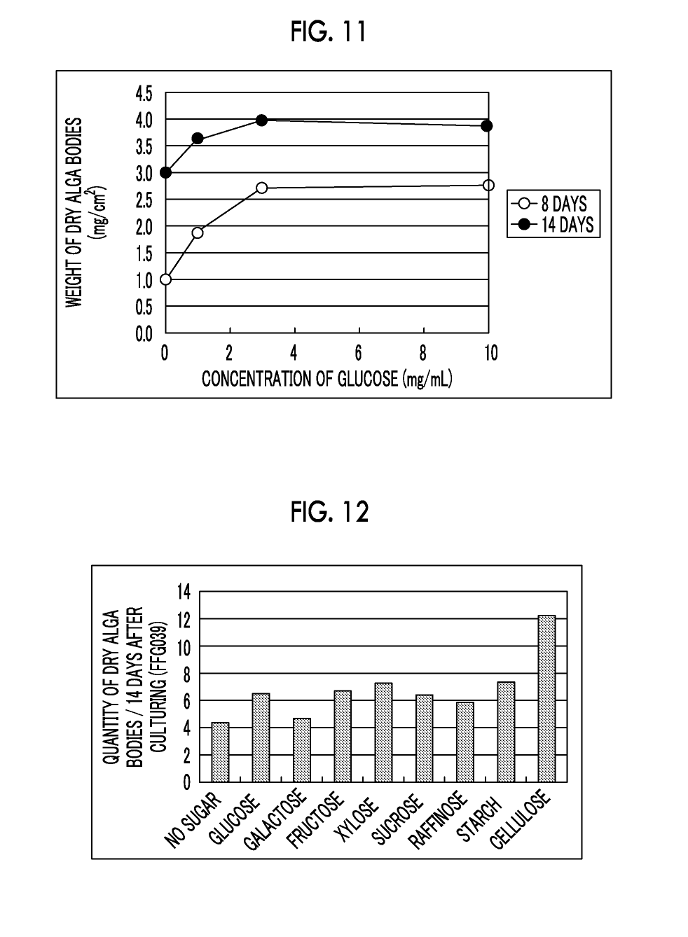

The results were shown in FIG. 11. The higher the concentration of glucose, the larger the quantity of dry alga bodies, and the quantity of dry alga bodies did not change at a concentration of greater than or equal to 3 mg/mL. That is, the amount of biomass was increased in accordance with the addition of sugar to the medium. This indicates that AVFF007 strains of microalgae can proliferate by having sugar as a nutrient source and that it is possible to expect an increase in the collected amount of algae by adding sugar to the medium.

### Example 6

### Execution Using Chlorococcum sp.

Pre-culture was performed through the same method as that in Example 5. However, as the species of algal bodies, FFG039 strains (concentration of algal bodies of 0.032 mg/mL which is equivalent to 5×10⁵ cells/mL) and AVFF007 strains (concentration of algal bodies of 5×10⁵ cells/mL) were used.

A sample was prepared through the same method as that in Example 5 to obtain a suspension liquid a of microalgae (FFG039 strains) and a suspension liquid b of microalgae (AVFF007).

These solutions were diluted and the turbidity was calculated by measuring the absorbance at 660 nm. 90 mL of a suspension liquid c at a concentration of 0.032 mg/mL was obtained by calculating the quantity of algal bodies of the above-described suspension liquid a from a relational expression between the turbidity and the number of algal bodies which have been calculated in advance and diluting the suspension liquid a with a CSiFF04 medium. As the medium, a CSiFF04 medium containing 10 mg/mL of sugar was used. Media were prepared with respect to sugar (monosaccharide (glucose, galactose, and fructose), monosaccharide.pentose (xylose), disaccharide (sucrose), trisaccharide (raffinose) polysaccharide (starch and cellulose)). In addition, similarly, a suspension liquid d with respect to the AVFF007 strains was obtained.

40 mL of the suspension liquid c containing FFG039 strains was put into a polystyrene case no. 28. The outer wall including the bottom surface was sealed with a black plate. The suspension liquid c was cultured under the same conditions as those in the pre-culture. A microalgal biofilm on the surface of water was collected through a deposition method using a nylon film 14 days after the culturing, and was freeze-dried. Then the quantity of dry alga bodies was measured. In addition, oil was extracted through hexane extraction. Similarly, a solution containing AVFF007 strains was prepared, and then, culturing, collecting, quantitative determination, and oil extraction were performed.

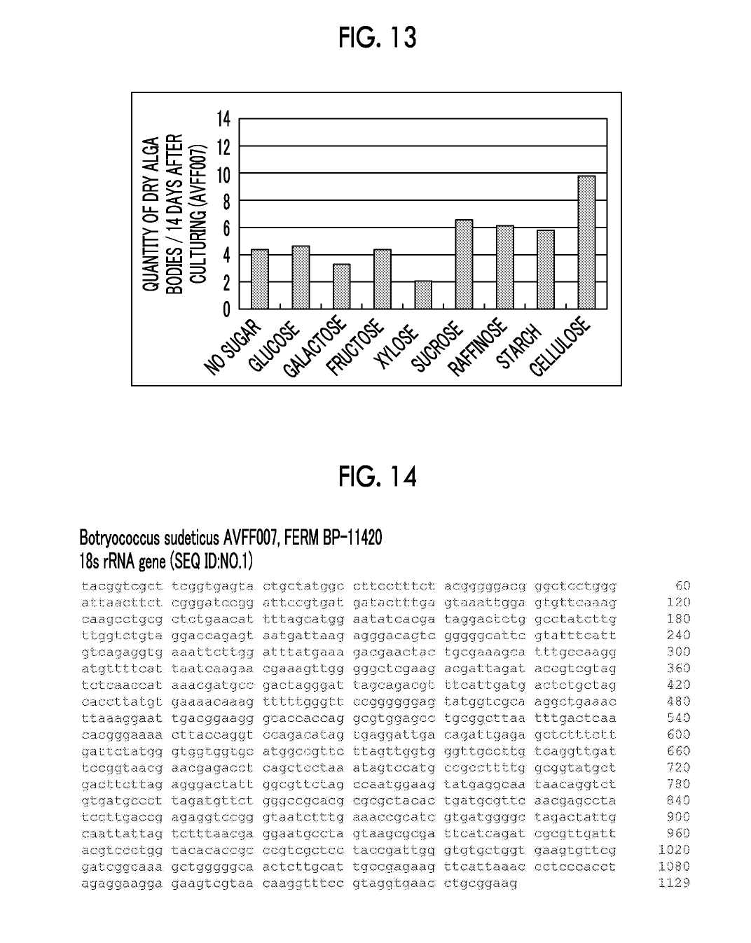

The results of the case of the FFG039 strains are shown in FIG. 12. The proliferation rate was improved in all of the cases of monosaccharide, disaccharide, trisaccharide, pentose, hexose, and polysaccharide, compared to the experimental conditions in which sugar is not added. Particularly, in a case of using cellulose as sugar, it was possible to confirm a significant improvement in the amount of proliferation. The results of the case of AVFF007 strains are shown in FIG. 13. In the case of AVFF007 strains, a decrease in the amount of proliferation was found in some kinds of sugar, but an increase in the amount of proliferation was found in the cases of disaccharide and polysaccharide. In the case of FFG039, the oil content was, in terms of dry weight proportion, 27.8 dry % when there is no sugar and 30 dry % to 35 dry % when there is sugar. In the case of AVFF007 strains, the oil content was, in terms of dry weight proportion, 19 dry % when there is no sugar and 20 dry % to 25 dry % when there is sugar. Dry % refers to an oil weight proportion per weight of the dry alga bodies.

From the above, it was found that it was possible to improve the amount of oil if a medium containing sugar was used.

### Example 7

### Case of Using Only Sugar without Using Light

Similarly to Example 5, pre-culture, preparation of a suspension liquid, and primary culture were performed. However, four samples which were irradiated with light and four samples which were not irradiated with light were prepared at a concentration of glucose of 10 mg/mL, and FFG039 strains were used as the species of algal bodies. In vacuum desiccators of the samples which were not irradiated with light, light was blocked using aluminum foil.

14 days after the culturing, similarly to Example 5, a microalgal biofilm on the surface of water was collected and the dry weight was measured. As a result, the dry weight in the samples which were irradiated with light and contain sugar in the media was 8.5 mg/cm². In contrast, the dry weight in the samples which were not irradiated with light and contains sugar in the media was 7.2 mg/cm².

From the above, it was found that FFG039 strains can proliferate if sugar was contained therein even if light was not used.

### Example 8

### Medium which Contains Sugar but No Nitrogen Compound

Pre-culture and first primary culture were performed through the same method as that in Example 7. However, all samples were irradiated with light.

14 days after the culturing, each medium was replaced with a CSiFF04 (N-) medium containing sugar. However, in four samples out of the prepared samples, a CSiFF04 (N-) medium containing no sugar was used.

After the replacement of the medium, second primary culture was performed for 7 days, and collection, freeze-drying, and oil extraction were performed through the same method as that in Example 7.

The quantity of dry alga bodies in a case where the nitrogen-limited medium containing sugar became 8.2 mg/cm² and the quantity of dry alga bodies in a case where the nitrogen-limited medium containing no sugar became 6.9 mg/cm². In addition, the oil content in each case became 38.7 dry % and 33.4 dry %.

From the above, with the use of the medium which contains sugar, but no nitrogen compound, the quantity of dry alga bodies was increased and the oil content was also improved.

### Example 9

A microalgal biofilm on the surface of water was collected similarly to Example 1-a. The collected amount was 4.83 mg/cm². The collection was performed several times through a deposition method using a nylon film while making the water depth 0.5 cm at a lower end of the nylon film.

After collecting a medium so as not to collect a microalgal biofilm on the bottom surface as possible, the total amount of the biofilm thereof was centrifugally separated and a supernatant was removed. Then, the dry weight of a residue was measured. As a result, the dry weight thereof became 0.08 mg/cm².

Microalgae on the bottom surface were collected using a cell scraper. The total quantity of microalgae was centrifugally separated and a supernatant was removed. Then, the dry weight of a residue was measured. As a result, the dry weight thereof became 2.82 mg/cm².

Microalgae adhered on the side surface of a culture vessel were not collected.

The similar test was performed on FFG039 strains, and as a result, each dry weight of the water surface algae, in the medium, and the bottom surface algae was 7.52 mg/cm², 0.13 mg/cm², and 2.57 mg/cm².

From the above, it was found that the quantity of algal bodies of the water surface algae and the bottom surface algae was greater than or equal to 98% of the quantity of algal bodies in the culture vessel except for the side surface of the culture vessel. In the case of these algal bodies, a medium containing glucose of 10 mg/mL was used.

### Example 10

The specific gravity of algal bodies of the sample obtained in Example 9 was measured through the following method. However, AVFF007 strains were used.

Cesium chloride was dissolved in a KOH solution (pH: 7.5) of 10 mM ethylenediaminetetraacetic acid (EDTA, ethylenediamine-N,N,N′,N′-tetraacetic acid) and 5 mM HEPES (4-(2-hydroxyethyl)-1-piperazineethanesulfonic acid) to prepare solutions with a cesium chloride concentration of 35% to 105% (w/v) at 10% intervals. Then, a concentration gradient was prepared such that the concentration became lower from a tip portion of a Pollyallomer tube (manufactured by Hitachi Koki Co., Ltd.) toward a liquid surface portion thereof.

AVFF007 strains at 5×10⁶ cells/mL were applied to the upper surface of the tube and centrifugal treatment was performed using a centrifuge for 30 minutes at a centrifugal force of 20000×g at 4° C.

The specific gravity of algal bodies floating on the liquid surface was within a range of 1.33 g/mL to 1.41 g/mL. In contrast, the specific gravity of algal bodies on the bottom surface was within a range of 1.41 g/mL to 1.48 g/mL.

From the above, it was found that the specific gravity of bottom surface algae was higher than that of algae on the liquid surface.

In the same manner as in Example 9, the similar test was performed using a medium containing sugar in a case of using FFG039 strains as the species of algal bodies. As a result, the specific gravity of the algal bodies on the liquid surface was within a range of 1.23 to 1.37 and the specific gravity of the bottom surface algae was within a range of 1.39 to 1.51.

### Example 11

Oil contents of the water surface algae and the bottom surface algae, which were obtained in Example 9, were measured through the same method as that described in Example 6. However, AVFF007 strains were used.

As a result, the oil content of water surface algae was 24.4% and the oil content of bottom surface algae was 15.3%.

From the above, it was found that the oil content of water surface algae was high.

In the same manner as in Example 9, the similar test was performed using a medium containing sugar in a case of using FFG039 strains as the species of algal bodies. As a result, the oil content of the algal bodies on the liquid surface was 34.6% and the oil content of the bottom surface algae was 28.5%.

### Example 12

The sizes (diameters) of the water surface algae and the bottom surface algae which have been obtained in Example 9 were measured while being observed with a microscope.

The sizes of 27 algal bodies were measured and an average value was used. However, AVFF007 strains were used.

The average size of water surface algae was 22.1 μm. The average size of bottom surface algae was 7.8 μm.

From the above, it was found that the sizes of the water surface algae were about three times larger than those of the bottom surface algae.

The similar test was performed using a medium containing sugar. As a result, the average size of algal bodies on the liquid surface was 21.7 μm and the size of bottom surface algae was 8.9 m.

### Example 13

A sample was obtained through the same method as that in Example 9. However, FFG039 strains were used.

The structure of a microalgal biofilm on the liquid surface was destroyed using forceps. As a result, a structure could be seen which was formed of a large number of bubbles therein. The structure destroyed using the forceps was picked up by the forceps and was put on a slide glass. Furthermore, a part of the bubble-like structure was transferred onto a slide glass through a transferring method.

The above-described two samples were set in a microscope, and each thickness thereof was measured using the difference in the focal distance between the surface of the glass and the surface of the microalgal biofilm. As a result, the thickness of the microalgal biofilm in the outside was 1.8 mm and the thickness in the inside was 0.2 mm.

From the above it was found that the three-dimensional structure of the microalgal biofilm on the liquid surface, in which a thick biofilm is formed outside the structure and a thin biofilm is formed inside thereof, was formed of a large number of bubbles.

The water surface algae which were cultured in a medium using sugar were also similarly observed, and as a result, the similar structure could be confirmed.

### Example 14

Culturing was performed similarly to Example 9. However, AVFF007 strains were used as a culture sample, and a structure of a microalgal biofilm on the liquid surface was observed on day 7.

As a result, a partially bubble-like structure was formed. The main structure was formed of a film-like structure of a two-dimensional structure. In addition, a pleat-like structure randomly entered the middle of the liquid surface from the film-like structure due to proliferation of the film-like structure.

The water surface algae which were cultured in a medium using sugar were also similarly observed, and as a result, the similar structure could be confirmed.

### Example 15

Culturing was performed through the same method as that in Example 9, and water surface algae were collected through a deposition method using a nylon film. When a part of the algae was slowly applied to the surface of water of a culture vessel into which a fresh medium was put, it is possible to make almost the entire quantity of the algae float on the liquid surface.

In contrast, there were almost no algal bodies floating on the liquid surface even if the above-described collected substance was put into a microtube into which a small amount of medium was put, pipetting was performed plural times, and the collected substance was slowly applied to the surface of water of a culture vessel into which a fresh medium was put.

When the culture vessels were performed through the same method as that in Example 9, a microalgal biofilm was formed on the liquid surface in both of the samples.

The water surface algae which were cultured in a medium using sugar were also similarly observed, and as a result, similar properties could be confirmed.

### Sequence Table Free Text

SEQ ID No: 1: part of base sequence of 18S rRNA gene of AVFF007 strains SEQ ID No: 2: part of base sequence of 18S rRNA gene of FFG039 strains

What is claimed is:

1. A culture method of microalgae having properties of producing useful substances, the method comprising:
a step of culturing microalgae in a medium within a culture vessel and forming a biofilm on the liquid surface of the medium; and
a step of changing the concentration of at least one component contained in the medium,
wherein the useful substances produced by microalgae were increased by changing the concentration of the component.

2. The culture method according to claim 1,
wherein the step of changing the concentration of the at least one component contained in the medium is performed by adding a liquid having a different composition from that of the medium, into the culture vessel or by removing a part or the entirety of the medium within the culture vessel and adding a liquid having a different composition from that of the medium.

3. The culture method according to claim 1,
wherein the step of changing the concentration of the at least one component contained in the medium is performed by decreasing the concentration of a component containing nitrogen or phosphorus.

4. The culture method according to claim 1,
wherein the removal or the addition of the medium refers to removal of the medium between a biofilm on the liquid surface and the bottom surface of the culture vessel or addition of the liquid having a different composition.

5. The culture method according to claim 1,
wherein, in the step of changing the concentration of the at least one component contained in the medium, the biofilm formed on the liquid surface is not removed.

6. The culture method according to claim 1, the method comprising:
a step of culturing microalgae in a medium within a culture vessel and forming a biofilm on the liquid surface of the medium;
a step of adding a liquid into the culture vessel; and
a step of collecting the biofilm from the culture vessel to which the liquid is added and the water depth of which is increased.

7. The culture method according to claim 1, further comprising:
a step of performing treatment of peeling an adhesion site between the biofilm and an inner wall of the culture vessel.

8. The culture method according to claim 1,
wherein the microalgae are green algae.

9. The culture method according to claim 1,
wherein the microalgae belong to Botryococcus sp., Chlamydomonas sp., Chlorococcum sp., Chlamydomonad sp., Tetracystis sp., Characium sp., or Protosiphon sp.

10. The culture method according to claim 1,
wherein the microalgae belong to the same species as that of Botryococcus sudeticus or Chlorococcum sp. FERM BP-22262.

11. The culture method according claim 1,
wherein the microalgae are Botryococcus sudeticus FERM BP-11420 or microalgae strains having taxonomically the same properties as those of Botryococcus sudeticus FERM BP-11420, or are Chlorococcum sp. FERM BP-22262 or microalgae strains having taxonomically the same properties as those of Chlorococcum sp. FERM BP-22262.

12. A method for manufacturing algal biomass, comprising:
a culture step including the culture method according claim 1; and
a step of collecting a formed biofilm.

13. The manufacturing method according to claim 12,
wherein the algal biomass is oil.

14. Microalgae which can form a biofilm on the liquid surface and have at least one characteristic selected from the group consisting of the following (1) to (8) when being cultured in a medium within a culture vessel:
(1) the sum of the quantity of algal bodies of microalgae existing on the liquid surface and in a region from 1 cm below the liquid surface to the liquid surface, and the quantity of algal bodies of microalgae on the bottom surface of a culture vessel is greater than or equal to 10 times the quantity of algal bodies existing in the other region within the culture vessel;
(2) the specific gravity of microalgae on the liquid surface is smaller than that of microalgae on the bottom surface of the culture vessel;
(3) the specific gravity of microalgae on the liquid surface is greater than that of water;
(4) the oil content of microalgae on the liquid surface is higher than that of microalgae on the bottom surface;
(5) the size of microalgae on the liquid surface is larger than that of microalgae on the bottom surface;
(6) a biofilm to be formed includes a film-like outer layer and an inner layer which has a plurality of bubble-like structures, and the outer layer is thicker than the inner layer;
(7) a part of a biofilm to be formed has a pleat-like structure in a medium; and
(8) in a case where microalgae obtained by collecting a formed biofilm and subjecting the collected biofilm to suspension treatment are seeded on the liquid surface of a medium, the microalgae can be deposited in the medium.

15. The microalgae according to claim 14,
wherein microalgae are green algae.

16. The microalgae according to claim 14,
wherein the microalgae belong to Botryococcus sp., Chlamydomonas sp., Chlorococcum sp., Chlamydomonad sp., Tetracystis sp., Characium sp., or Protosiphon sp.

17. The microalgae according to claim 14,
wherein the microalgae belong to the same species as that of Botryococcus sudeticus or Chlorococcum sp. FERM BP-22262.

18. The microalgae according to claim 14, of which the identity with base sequences of a partial region corresponding to Chlorococcum sp. RK261 among base sequences encoding a gene region of 18S rRNA is 95.00% to 99.99% or which belong to Chlorococcum sp.,
wherein the 18S rRNA gene thereof has sequence identity of at least 99.94% with polynucleotide formed of a base sequence of SEQ ID No: 2.

19. The microalgae according to claim 14, being microalgae which are Chlorococcum sp. FFG039 strains (accession number of FERM BP-22262) or microalgae having taxonomically the same properties as those of Chlorococcum sp. FFG039 strains.
